(19) United States
(12) Patent Application Publication
(10) Pub. No.: US 2022/0139545 A1
(43) Pub. Date: May 5, 2022

(54) Systems and Methods for On-Device Real-Time Access and Review of Events during a Patient Treatment Episode

(71) Applicant: Physio-Control, Inc., Redmond, WA (US)

(72) Inventors: Michelle Liu, Sammamish, WA (US); Mark Stamnes, Redmond, WA (US); Sarah Mynhier, Redmond, WA (US)

(21) Appl. No.: 17/499,963

(22) Filed: Oct. 13, 2021

Related U.S. Application Data
(60) Provisional application No. 63/107,778, filed on Oct. 30, 2020.

(51) Int. Cl. G16H 40/63; G16H 20/10; G16H 10/60; A61N 1/39

(52) U.S. Cl. CPC G16H 40/63; G16H 20/10; G06F 3/0485; A61N 1/3904; A61N 1/3993; G16H 10/60

(57) ABSTRACT

An example method includes detecting events that occur during the on-going patient treatment; for each event detected: capturing in real-time physiologic parameters of the patient at a point in time at which the event occurs, generating a waveform comprising a first portion of data before the event and a second portion of data after the event generating an event record including temporal information of when the event has occurred, identification of the event, the physiologic parameters at a time when the event occurs, and the waveform; generating a display of an events list comprising a scrollable list of respective events records associated with the detected events, each event record showing respective temporal information, respective identification of a respective event, respective physiologic parameters, and respective waveforms such that a healthcare professional has access to the events records throughout the on-going patient treatment.

### CROSS REFERENCE TO RELATED APPLICATION

The present application claims priority to U.S. Provisional Application No. 63/107,778 filed on Oct. 30, 2020, the entire contents of which are herein incorporated by reference as if fully set forth in this description.

### BACKGROUND

During an emergency episode (e.g., cardiac arrest or arrhythmia), a defibrillator, such as an automated external defibrillator (AED), can provide potentially lifesaving defibrillation treatment. For instance, a defibrillator is configured to supply a charge through the patient's heart via a set of defibrillation pads of a therapy cable to restore a normal heartbeat.

During the emergency episode, while a defibrillator is attached to a patient, several events occur. For instance, a healthcare professional, e.g., a physician or an Emergency Medical Technician (EMT), may administer medications or apply treatments (e.g., apply an electric shock) during the episode. Currently, healthcare professionals do not have real-time access to records of events that have occurred during the episode. Thus, the healthcare professional may forget what treatments or medications were administered a few minutes earlier.

Further, if there is a hand-off of the patient from one healthcare professional to another (e.g., from and EMT to hospital staff) during an on-going episode, a hot-debrief occurs to discuss the patient state. In the hot-debrief, the healthcare professional who has been treating the patient provides information to the receiving healthcare professional. Particularly, the healthcare professional who has been treating the patient tries to remember all the events that have occurred during the episode to provide information about such events to the receiving healthcare professional. However, it is not uncommon that the treating personnel forget details and events that have occurred, and the receiving healthcare may miss critical information about the state of the patient without access to all the events that have occurred.

### SUMMARY

Within examples described herein, systems and methods for on-device real-time access and review of events during a patient treatment episode.

Within additional examples described herein, systems and methods are described that relate to providing an on-device real-time patient events review tools with physiologic parameters (e.g., vital signs) and waveform review capabilities, thus providing an on-device presentation of collected data and making the data available immediately during an emergency episode.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The novel features believed characteristic of the illustrative examples are set forth in the appended claims. The illustrative examples, however, as well as a preferred mode of use, further objectives and descriptions thereof, will best be understood by reference to the following detailed description of an illustrative example of the present disclosure when read in conjunction with the accompanying Figures.

FIG. 1 illustrates an example defibrillation scene, in accordance with an example implementation.

FIG. 2 illustrates a perspective view of a defibrillator, in accordance with an example implementation.

FIG. 3 illustrates a block diagram of the defibrillator in FIG. 2, in accordance with an example implementation.

FIG. 4 illustrates a graphical user interface, in accordance with an example implementation.

FIG. 5 illustrates a care record window that is displayed when a collapsed menu button on the graphical user interface is pressed, in accordance with an example implementation.

FIG. 6 illustrates an events list view pane that is displayed when an Events List tab on the graphical user interface is selected, in accordance with an example implementation.

FIG. 7 illustrates an events list when a Treatments tab is selected, in accordance with an example implementation.

FIG. 8 illustrates the events list when a Medications tab is selected, in accordance with an example implementation.

FIG. 9 illustrates the events list when a Generic events tab is selected, in accordance with an example implementation.

FIG. 10 illustrates the events list when a 12/15 Lead tab is selected, in accordance with an example implementation.

FIG. 11 illustrates an events menu that appears when an Events button is selected, in accordance with an example implementation.

FIG. 12 illustrates the events menu when a Quick Events option is selected, in accordance with an example implementation.

FIG. 13 illustrates the events menu when a Quick Buttons option is selected, in accordance with an example implementation.

FIG. 14 illustrates a partial view of the graphical user interface showing a reminder display, in accordance with an example implementation.

FIG. 15 illustrates a partial view of the graphical user interface showing a notification of an added event, in accordance with an example implementation.

FIG. 16 illustrates the graphical user interface with waveforms associated with a shock event being displayed, in accordance with an example implementation.

FIG. 17 illustrates horizontal scrolling of waveforms, in accordance with an example implementation.

FIG. 18 is a flowchart of a method for operating a defibrillator, in accordance with an example implementation.

FIG. 19 is a flowchart of additional operations that are executable with the method of FIG. 18, in accordance with an example implementation.

FIG. 20 is a flowchart of additional operations that are executable with the method FIG. 18, in accordance with an example implementation.

FIG. 21 is a flowchart of additional operations that are executable with the method FIG. 18, in accordance with an example implementation.

FIG. 22 is a flowchart of additional operations that are executable with the method FIG. 18, in accordance with an example implementation.

FIG. 23 is a flowchart of additional operations that are executable with the method FIG. 18, in accordance with an example implementation.

FIG. 24 is a flowchart of additional operations that are executable with the method FIG. 18, in accordance with an example implementation.

FIG. 25 is a flowchart of additional operations that are executable with the method FIG. 18, in accordance with an example implementation.

FIG. 26 is a flowchart of additional operations that are executable with the method FIG. 18, in accordance with an example implementation.

FIG. 27 is a flowchart of additional operations that are executable with the method FIG. 18, in accordance with an example implementation.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

Currently, when a defibrillator is applied to a patient in an emergency episode (e.g., a cardiac arrest or arrhythmia) in the field, the defibrillator gathers data associated with various events that occur during the emergency episode. That data provide unique, valuable insight into the cause of the emergency heart episode and can help a physician or other healthcare professional select a course of care for the patient. Often, however, during an emergency episode involving several events (treatments, medications, alarms, etc.) happening quickly, a healthcare professional might not remember all the events that have occurred and might not have time to document all such events.

In other situations, one healthcare professional, e.g., a paramedic or EMT, may care for a patient for a portion of an episode, and then transfers the patient to a hospital for another healthcare profession, e.g., physician, to continue caring for the patient. The physician asks several questions about the condition of the patient such as initial heart rhythm, how many shocks have been applied, how many doses of a particular medication have been administered, etc. The paramedic tries to recall from memory or written-down notes all the events that have occurred during the episode and may miss some events.

Thus, for several reasons, it is a common problem that data about events that have occurred during an emergency episode might not make it to a physician, and the patient might therefore not receive appropriate care. For example, if the physician in the hospital does not know that a particular medication has been administered or a treatment (e.g., Airway or shock) has been applied to the patient, the physician may prematurely apply the same medication or treatment. Thus, currently, there is no way for a receiving physician to have access to accurate information related to all the events that have occurred during an on-going emergency episode.

It may thus be desirable to provide a healthcare professional with real-time access to accurate information about all events that occur during an on-going episode. The term “real-time” is used throughout herein to indicate any time during care for patient having an on-going emergency episode, while the device (e.g., the defibrillator) continues to operate as intended (e.g., capture events, apply shocks, etc.). Also, “events” include medications administered, treatments applied, any generic event that might occur, physiologic alarms (heart rate increased beyond a threshold), physiologic parameters (e.g., vital sign) sets, electrocardiogram (ECG) reports (e.g., 12/15 Lead ECG reports), and therapies applied (e.g., electric shocks delivered).

Example methods and systems describe providing an on-device real-time patient event review tools with physiologic parameters (e.g., vital signs) and waveform review capabilities, thus providing an on-device presentation of collected data and making the data available immediately during an emergency episode. This way, a treating healthcare professional has continual access to history, medications doses, or any other events that has occurred with time stamps of each event in addition to various physiologic parameters and waveforms (e.g., signals from sensors) that have been captured during the event. Thus, a healthcare professional need not remember all the events or document the events while caring for the patient. Further, such methods and systems may help ease cognitive off-load of a paramedic or EMT through the handing-off or transition to a hospital or other treating facility.

Additional example methods and systems describe detecting that an event has occurred or receiving information that the event has occurred, and then capturing various physiologic parameters when the event occurs, obtaining real-time data indicating variation of one or more physiologic parameters (ECG, oxygen, blood pressure, etc.) before the event (e.g., within a time window of a particular period of time before the event such as 3-5 seconds), obtaining real-time data indicating variation of the one or more physiologic parameters after the event (e.g., within a time window of a particular period of time after the event such as 8 seconds), rendering respective waveforms of the one or more physiologic parameters, and attaching or associating the respective waveforms to the event record. This way, when a healthcare professional reviews a particular event, the healthcare professional have access to values of the physiologic parameters as well as waveforms that show the effect of the event on the patient's.

Additional example methods and systems describe generating display of a scrollable and selectable list of event records of all the events that have occurred during an on-going episode. Each event record includes information identifying the event (e.g., indicating the name of the event), temporal information of when the event has occurred (e.g., a time stamp or chronological time of the event and time elapsed since the event has occurred), various physiologic parameters captured when the event occurs, waveforms of physiologic parameters (e.g., ECG, blood pressure, etc.) before and after the event, a timer indicating a count-down to a time where a medication or treatment is due to be re-administered, among other information. In an example, events list can be filtered by the type of events, e.g., treatments, medications, generic events, or 12/15 Lead reports, alarms, etc.

When an event is selected from the scrollable list, the associated signals or waveforms are displayed. In an example, the waveforms are scrollable (e.g., horizontally-scrollable) to navigate the waveform over a particular period of time (e.g., 11 seconds).

Providing access to such events, event records, and associated information in such manner facilitates providing timely, informed, and appropriate decision making and transition of care.

Further details and features of these methods and systems are described hereinafter with reference to the figures.

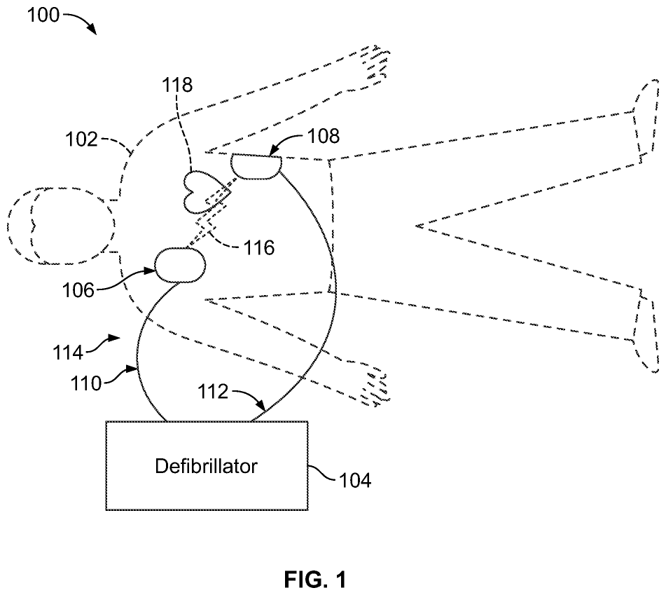

Referring now to the figures, FIG. 1 illustrates an example defibrillation scene 100. As shown in FIG. 1, a patient 102 is lying on their back. Patient 102 could be a patient in a public space, a home, a pre-hospital environment (e.g., an emergency ambulance), or a hospital. A defibrillator 104 is being used to treat patient 102. As shown in FIG. 1, defibrillation pads 106, 108 of defibrillator 104 are applied to a chest of patient 102. Defibrillation pad 106 is coupled to defibrillator 104 via an electrode lead 110. Defibrillation pad 108 is coupled to defibrillator 104 via an electrode lead 112. Defibrillation pads 106, 108 and electrode leads 110, 112 are collectively referred to as a therapy cable 114. Defibrillator 104 can be used to deliver, via therapy cable 114, a shock 116. Shock 116 can go through a heart 118 of patient 102, in an attempt to restart heart 118 or restore normal heart rhythm.

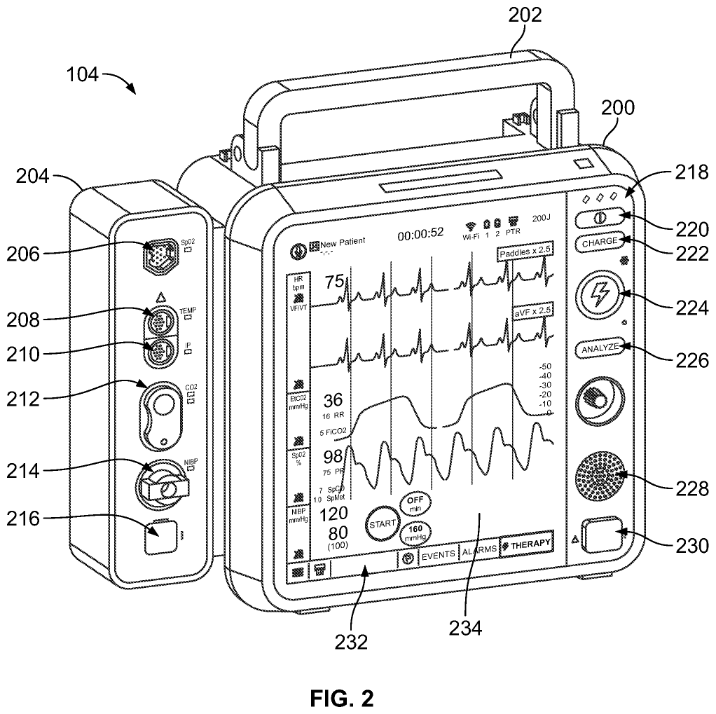

FIG. 2 illustrates a perspective view of the defibrillator 104, in accordance with an example implementation. Defibrillator 104 can be one of multiple different types, each with different sets of features and capabilities. As one example, defibrillator 104 can be an AED An AED can make a decision as to whether or not to deliver a shock to a patient automatically. For example, an AED can sense physiologic conditions, such as shockable heart rhythms, of a patient via defibrillation pads applied to the patient, and make the decision based on an analysis of the patient's heart. Further, an AED can either deliver the shock automatically, or instruct a user to deliver a shock, e.g., by pushing a button.

The defibrillator 104 described herein is a monitor defibrillator. Monitor defibrillators are intended to be used by trained medical professionals, such as doctors, nurses, paramedics, emergency medical technicians, etc. As the name suggests, a monitor defibrillator is a combination of a monitor and a defibrillator.

As a defibrillator, a monitor defibrillator can be one of different varieties, or even versatile enough to be able to switch among different modes that individually correspond to the varieties. One variety is that of an automated defibrillator, which can determine whether a shock is needed and, if so, charge to a predetermined energy level and instruct the user to deliver the shock. Another variety is that of a manual defibrillator, where the user determines whether a shock is needed and controls delivery of the shock. As a patient monitor, the monitor defibrillator has features additional to what is needed for operation as a defibrillator. These features can be for monitoring physiologic indicators of a patient in an emergency scenario, for instance.

The defibrillator 104 has a housing 200 and a handle 202 to facilitate moving the defibrillator 104. The defibrillator 104 includes an input module 204 coupled to or integral with the housing 200. The input module 204 includes various ports that can be connected to various sensors to receive input information indicative of various physiologic parameters of the patient being treated and monitored.

For example, the input module 204 includes a port 206 configured to be connected to an oxygen saturation (SpO2) sensor, port 208 configured to be connected to a temperature sensor, port 210 configured to be connected to a sensor configured to measure invasive blood pressure (IP) via a catheter, port 212 configured to be connected to a sensor configured to measure of partial pressure of carbon dioxide (CO2) in gases in the airway via capnography, port 214 configured to be connected to a non-invasive blood pressure (NIBP) sensor, among other physiologic parameters. The defibrillator 104 includes a communication port 216 such as a Universal Serial Bus (USB) port that can be used, for example, to connect input devices (mouse, keyboard) to the defibrillator 104.

The housing 200 also includes a therapy cable port (not shown, e.g., on the opposite side of the housing 200 relative to the input module 204). The therapy cable 114 is connects to the defibrillator 104 via the therapy cable port, such that the defibrillator 104 can apply shocks and received heart rate (HR) and ECG data of the patient.

The defibrillator 104 includes a user interface 218. The user interface 218 can take any of a number of forms. For example, the user interface includes a physical user interface (e.g., physical buttons, knobs, etc.) and a graphical user interface (GUI) 232 that allows a healthcare professional to interact with and operate the defibrillator 104.

The user interface 218 may include input devices for receiving inputs from users and output devices to provide information to the user. Such input devices may include various controls, such as pushbuttons, keyboards, touchscreens, a microphone, a fingerprint scanner, a retinal scanner, and/or a camera, etc.

For example, the user interface 218 includes a power button 220 to turn the defibrillator 104 on and off (e.g., “On-Off” button), a charge button 222 that causes the defibrillator 104 to build an electric charge to be applied to the patient, a defibrillation shock button 224 that causes the defibrillator 104 to apply a therapy shock to a patient during a fibrillation episode, and an analyze button 226 that causes a processor of the defibrillator 104 to analyze patient data (e.g., ECG data) to facilitate determining the appropriate time to apply a shock, for example.

The user interface 218 also includes output devices, which can be visual, audible or tactile, for communicating to a user, such as speaker 228. An output device can be configured to output a warning or alarm, which warns or instructs the healthcare professional regarding a physiologic parameter of the patient or regarding due time for a treatment or medication. The user interface 218 can also include a USB output port 230 to facilitate connecting the defibrillator 104 to an output device such as a printer, for example.

The defibrillator 104 has a touchscreen 234 to display the GUI 232, which can show what is detected and measured, provide visual feedback to the healthcare professional about condition of the patient, and allow the healthcare professional to interact with and operate the defibrillator 104. Particularly, the touchscreen 234 is a display device, which allows the healthcare professional to interact with the defibrillator 104 by touching areas on the GUI 232 displayed on the touchscreen 234.

As described in more detail below, the GUI 232 has multiple visual user interface items that are selectable or “clickable” by the healthcare professional including user-selectable icons, user-selectable on-screen buttons, menus, widgets, scroll bars, graphical objects, and other items for facilitating user interaction.

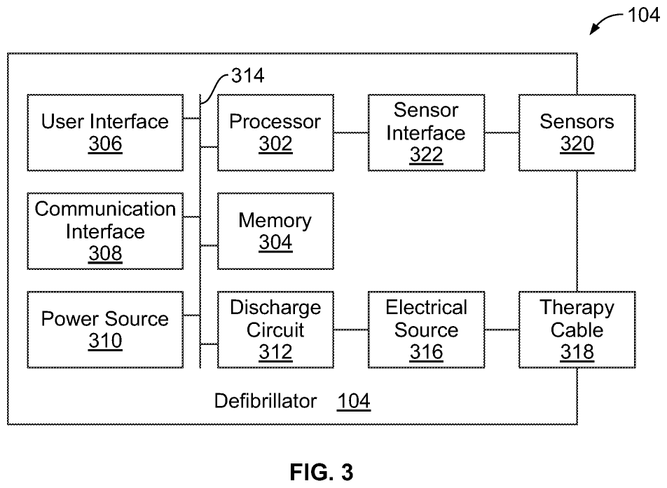

FIG. 3 illustrates a block diagram of the defibrillator 104, in accordance with an example implementation. The defibrillator 104 includes a processor 302, a memory 304, user interface 306 (e.g., the user interface 218), a communication interface 308, a power source 310, and a discharge circuit 312, each connected to a communication bus 314. The defibrillator 104 also includes an electrical source 316 connected to discharge circuit 312 and to a therapy cable 318 (e.g., therapy cable 114).

Memory 304 may include one or more computer-readable storage media that can be read or accessed by processor 302. The computer-readable storage media can include volatile and/or non-volatile storage components, such as optical, magnetic, organic or other memory or disc storage, which can be integrated in whole or in part with processor 302. The non-transitory data storage is considered non-transitory computer-readable media. In some examples, the non-transitory data storage can be implemented using a single physical device (e.g., one optical, magnetic, organic or other memory or disc storage unit), while in other examples, the non-transitory data storage can be implemented using two or more physical devices.

The non-transitory data storage thus is a non-transitory computer-readable medium, and executable instructions are stored thereon. The executable instructions include computer executable code that can be executed by the processor 302.

Processor 302 may include a general-purpose processor or a special purpose processor (e.g., digital signal processor, application specific integrated circuit, graphics processing unit, etc.). Processor 302 may receive inputs from other components of defibrillator 104 and process the inputs to generate outputs that are stored in the non-transitory data storage or displayed on the touchscreen 234. Processor 302 can be configured to execute instructions (e.g., computer-readable program instructions) that are stored in the non-transitory data storage and are executable to provide the functionality of the defibrillator 104 described herein.

The user interface 306 represents the user interface 218 described above with respect to FIG. 2.

Communication interface 308 may be one or more wireless interfaces and/or one or more wireline interfaces that allow for both short-range communication and long range communication to one or more networks or to one or more remote devices. Such wireless interfaces may provide for communication under one or more wireless communication protocols, such as Bluetooth, Wi-Fi (e.g., an institute of electrical and electronic engineers (IEEE) 802.11 protocol), Long-Term Evolution (LTE), cellular communications, near-field communication (NFC), radio-frequency identification (RFID), and/or other wireless communication protocols. Such wireline interfaces may include an Ethernet interface, USB interface (e.g., including communication port 216 and USB output port 230), or similar interface to communicate via a wire, a twisted pair of wires, a coaxial cable, an optical link, a fiber-optic link, or other physical connection to a wireline network. Communication interface 308 thus may include hardware to enable communication between defibrillator 104 and other devices (not shown). The hardware may include transmitters, receivers, and antennas, for example.

Power source 310 may include battery power, or a wired power means such as an AC power connection.

Electrical source 316 can be configured to store electrical energy in the form of an electrical charge, when preparing for delivery of a shock. Discharge circuit 312 can be controlled by the processor 302 to permit the energy stored in electrical source 316 to be discharged to defibrillation pads (e.g., defibrillation pads 106, 108) of therapy cable 318 (e.g., therapy cable 114) automatically, or when the defibrillation shock button 224 is pressed, for example. Discharge circuit 312 can include one or more switches, such as an H bridge.

Processor 302 can instruct discharge circuit 312 to output a shock using one of various energy levels. The energy levels can range from 50 Joules to 360 Joules. For instance, for an adult, processor 302 can select an energy level from an adult energy sequence that includes energy levels of 200 Joules, 300 Joules, and 360 Joules. Whereas, for a pediatric patient, processor 302 can select an energy level from a pediatric energy sequence that includes energy levels of 50 Joules, 75 Joules, and 90 Joules.

Therapy cable 318 can be detachable from the housing 200 of the defibrillator 104 by way of a connector. The connector can be a tabbed, male connector that is compatible with a port of the defibrillator 104. The defibrillation pads of therapy cable 318 can be similar to defibrillation pads 106, 108 of FIG. 1. The defibrillation pads can include sensors that provide physiologic monitoring data measurements to processor 302. For example, the defibrillation pads can include sensors that measure HR and heart electrical activity such as ECG.

As described in more detail below, the processor 302 is configured to detect various events during a patient care episode or receive information indicative of events, and responsively generate in real-time an event record for each event, where the event record is retrievable in real-time by healthcare professional during the episode. The event record includes temporal information about when the event occurs, various physiologic parameters captured when the event has occurred, and one or more waveforms of particular physiologic parameters (e.g., HR, blood pressure, ECG, etc.) that shows variation of the particular physiologic parameters before and after the event.

For example, after a shock is delivered (i.e., after a shock event occurs), or in parallel with the instructing of discharge circuit 312 to deliver a shock, processor 302 can store data indicative of the shock in memory 304. The data indicative of the shock can include one or any combination of an energy level of the shock, a timestamp associated with the shock, an indication of a number of the shock (e.g., an indication that the shock is the first shock, second, shock, third shock, etc.), an error code associated with the shock, and a signal or waveform that shows HR or ECG before and after the event.

In another example, during a patient care event, processor 302 can detect the event of return of spontaneous circulation (ROSC) after delivering a shock. Processor 302 determines that ROSC has been achieved using one or more of the following techniques: inferring that ROSC has been achieved via electrical signals; detecting a motion artifact that does not correspond to compressions or moving a patient; determining whether a trend after serval complete PQRST waveforms shows degradation; identifying respiratory breath from ECG; receiving information (e.g., wirelessly) from an accessory configured to deliver information to defibrillator 104, such as blood pressure, SpO2, CO2, etc.; voice recognition that identifies keywords such as “I feel a pulse!.” Processor 302 can also determine that ROSC is achieved after delivering a shock based on receiving an indication from another device. For instance, processor 302 can send data obtained by defibrillator 104 to a server in network. The server, in turn, can analyze the data to determine whether or not the data is indicative of ROSC being achieved (e.g., using any of the techniques noted above), and send to defibrillator 104 data indicative of whether or not ROSC has been achieved.

In another example, processor 302 can analyze ECG data, determine a fibrillation type using the ECG data, and store an indication of the fibrillation type. Ventricular fibrillation (VF) can be qualified as either refractory VF or recurrent VF. Refractory VF refers to VF that persists despite shock delivery. This is in contrast to recurrent VF, which is VF that re-appears after it had previously been terminated. The indication of fibrillation type could therefore include an indication of refractory VF or an indication of recurrent VF. Similarly, processor 302 can analyze ECG data, determine a coarseness of a VF waveform, and store an indication of the coarseness of the VF waveform. As still another example, processor 302 can store an initial rhythm measured by defibrillator 104, such as a few seconds of raw ECG data that is obtained before delivery of any shocks. Processor 302 can also determine and store data indicative of an algorithm used to measure the initial rhythm, such as data indicative of a name of the algorithm. In some examples, processors 302 can analyze ECG data and determine an amplitude spectrum area (AMSA) using the ECG data.

As yet another example, processor 302 can determine whether cardiopulmonary resuscitation (CPR) is being performed, and then store in memory 304 data indicative of whether or not CPR was performed on the patient. For example, processor 302 can determine whether CPR is being performed based on analysis of impedance signals received from the defibrillation pads of therapy cable 318. As another example, processor 302 can determine whether CPR is being performed based on an analysis of an ECG signal. CPR results in a rhythmic change in ECG signal. Processor 302 can detect such a change using signal processing. Such processing can involve providing the ECG signal to a trained neural network that is configured to output an indication of whether the ECG signal is indicative of CPR being performed. The neural network can be trained using ECG signals that are known to have been captured while CPR is being performed. The data indicative of whether or not CPR was performed can include data for individual compressions (e.g., compression rate data). Additionally or alternatively, the data indicative of whether or not CPR has been performed can include a binary indication (e.g., yes or no), or a qualitative indication (e.g., no CPR; bad CPR; moderate CPR; good CPR; great CPR). Processor 302 can also determine and store in memory 304 data indicative of whether or not defibrillator 104 advised a healthcare professional to continue CPR after a shock was delivered.

In addition to detecting some events automatically, the processor 302 can also receive information via the GUI 232 of the defibrillator 104 indicative of occurrence of events. For instance, as described below, a healthcare professional can use the user-interface items on the touchscreen 234 to input information regarding a particular event (e.g., a treatment or medication administered to the patient). The term “automatically” is used throughout herein to indicate the defibrillator 104 or the processor 302 programmatically (e.g., through execution of instructions) performing an action/operation based on a certain trigger event occurring. In this way, the defibrillator 104 or the processor 302 automatically performs the operation without user input to initiate the action/operation.

The defibrillator 104 can further include physiologic monitoring sensors 320 and a sensor interface 322 (e.g., the input module 204) that couples physiologic monitoring sensors 320 to processor 302. Physiologic monitoring sensors 320 allow for monitoring physiologic indicators of a patient. Any number or type of sensors may be used depending on treatment or monitoring of the patient. In many instances, a variety of sensors are used to determine a variety of physiologic monitoring data. Physiologic monitoring data can include vital sign data (e.g., HR, respiration rate, blood pressure, body temperature, ECG data, etc.), as well as signals from other sensors described herein. In addition, physiologic monitoring data can also include treatment monitoring data, such as location at which an endotracheal tube has been placed or other sensor context information. The physiologic monitoring data can include timestamps associated with a time of collection and may be considered a measurement at a specific time. In some instances herein, physiologic monitoring data refers to one measurement and data associated with the one measurement, and in other instances, physiologic monitoring data refers to a collection of measurements as context indicates.

Physiologic monitoring sensors 320 can include sensors that measure heart electrical activity such as ECG, saturation of the hemoglobin in arterial blood with (SpO2), carbon monoxide (carboxyhemoglobin, COHb) and/or methemoglobin (SpMet), partial pressure of carbon dioxide (CO2) in gases in the airway by means of capnography, total air pressure in the airway, flow rate or volume of air moving in and out of the airway, blood flow, blood pressure such as non-invasive blood pressure (NIBP) or invasive blood pressure (IP) by means of a catheter, core body temperature with a temperature probe in the esophagus, oxygenation of hemoglobin within a volume of tissue (rSO2), indicating level of tissue perfusion with blood and supply of oxygen provided by that perfusion, and so forth.

Outputs, e.g., signals, from physiologic monitoring sensors 320 are conveyed to processor 302 by way of sensor interface 322. Processor 302 records the signals and attaches them to the event record, which can be retrieved by the healthcare professional in real-time during an on-going patient episode.

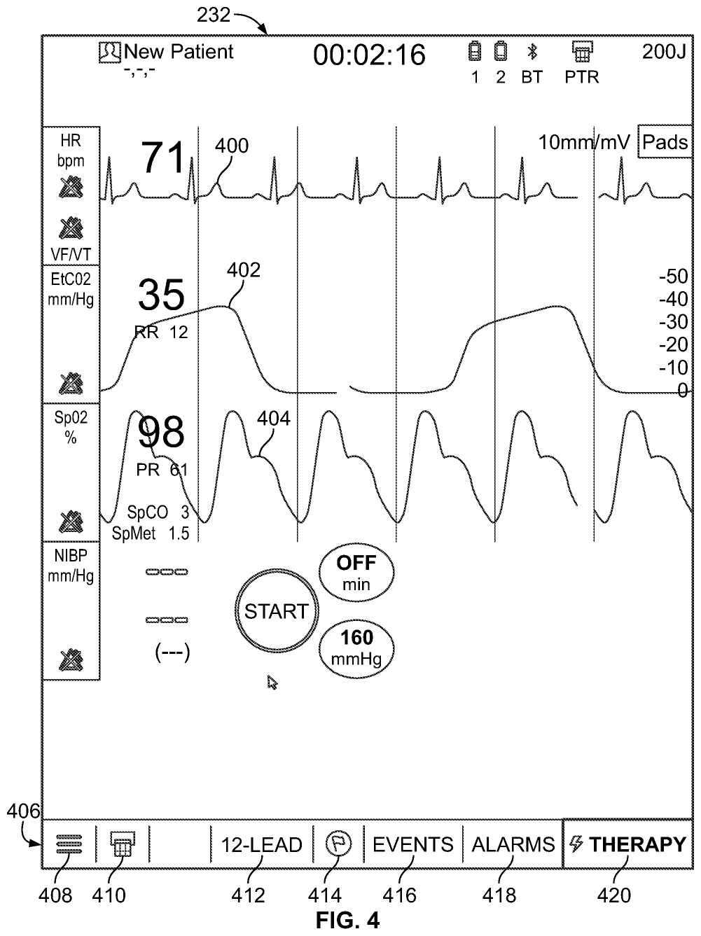

FIG. 4 illustrates the GUI 232, in accordance with an example implementation. The processor 302 is configured to generate a display of or visually present the GUI 232 on the touchscreen 234 to allow healthcare professionals to interact with the defibrillator 104 through user-selectable on-screen graphical items (e.g., buttons, menus, widgets, scroll bars, graphical objects, audio indicators, icons, etc.) to facilitate user-interaction. The processor 302 generates the display of the GUI 232 on the touchscreen 234, and the healthcare professional can then select the user-selectable user-interface items by pressing or selecting areas on the touchscreen 234 displaying the items.

The GUI 232 also shows patient data including physiologic parameters and waveforms, etc. output or processed by the processor 302 as well as provided by the physiologic monitoring sensors 320. The touchscreen 234 thus operates as both an input device and output device and is layered on the top of an electronic visual display of the defibrillator 104.

The GUI 232 includes interactive visual components or objects that convey information and represent actions that can be taken by the healthcare professional. The objects can change color, size, or visibility when the user interacts with them. The GUI objects include icons, menus, and buttons. These graphical objects can be enhanced with sounds, or visual effects like change in color, transparency, or drop shadows to facilitate interaction with the GUI 232.

As shown in FIG. 4, when the defibrillator 104 is connected or attached to a patient, the GUI 232 displays waveforms next to a side rectangle having a particular color and labelled by the physiologic parameter to which the waveform pertains. For example, the GUI 232 includes waveform 400 for HR, waveform 402 for End-tidal CO2 (EtCO2), which indicates the partial pressure or maximal concentration of carbon dioxide (CO2) at the end of an exhaled breath, and waveform 404 for SpO2. The GUI 232 can also display NIBP values for the patient.

The GUI 232 has a taskbar or main menu 406 at the bottom having different tabs and menu options. Particularly, the GUI 232 has collapsed menu button 408, print button 410, 12-Lead button 412, Generic Event button 414, Events button 416, Alarms button 418, and Therapy button 420.

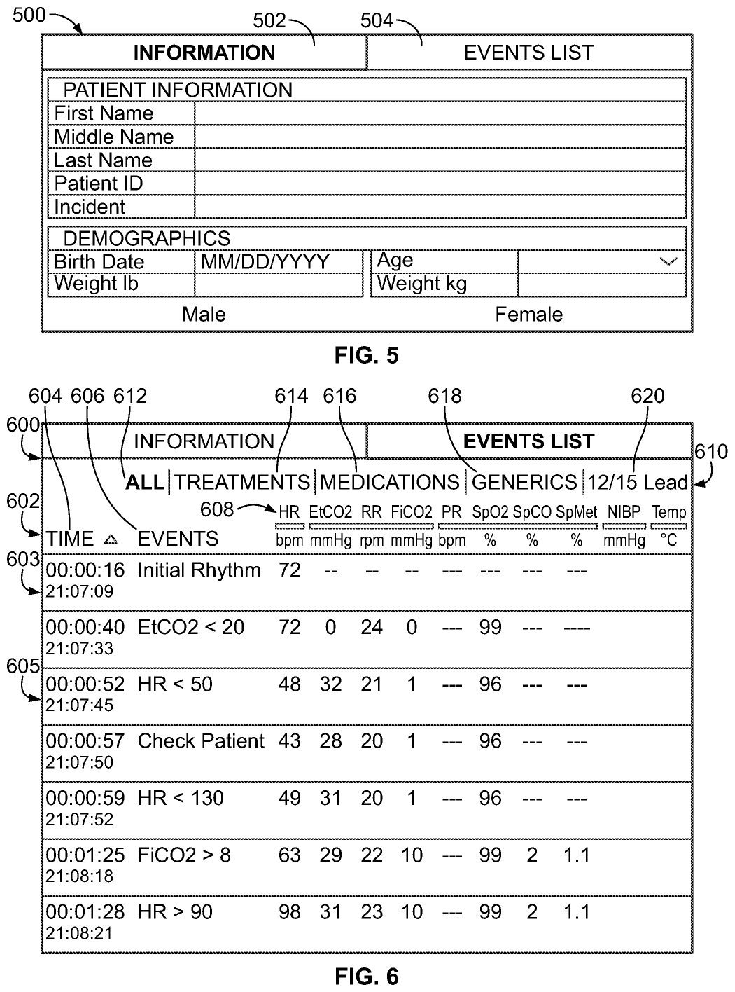

FIG. 5 illustrates a care record window 500 that is displayed when the collapsed menu button 408 is pressed, in accordance with an example implementation. The care record window 500 has two tabs: an Information tab 502 and an Events List tab 504. When the Information tab 502 is selected, the patient information appears and the healthcare professional can enter information for a new patient such as name, age, gender, and weight.

FIG. 6 illustrates an events list view pane 600 that is displayed when the Events List tab 504 is selected, in accordance with an example implementation. When the Events List tab 504 is selected, an events list 602 is displayed that includes a scrollable list of events records of events that have occurred during the current on-going patient episode (e.g., during a cardiac arrest or arrhythmia episode).

The events list 602 includes multiple rows and each row represents an event record such as Initial Rhythm event record 603 and “HR<50” event record 605, etc. The event records are listed in chronological order such that the healthcare professional can navigate the events chronologically. They can be listed in an ascending or descending chronological order as desired.

The events list 602 has several columns including time column 604 indicating both the time elapsed since the event has occurred and chronological time when the event has occurred. An events column 606 shows the name of the event. To the right of each event name, the event list 602 shows multiple physiologic parameter columns 608, each column having a value of a physiologic parameter (e.g., a vital sign) monitored and captured at the time of the event. For example, the physiologic parameters listed in the physiologic parameter columns 608 include HR, EtCO2, respirator rate (RR), Fractional Concentration of Inspired CO2 (FiCO2), pulse rate (PR), SpO2, SPCO, SpMet, NIBP, and temperature.

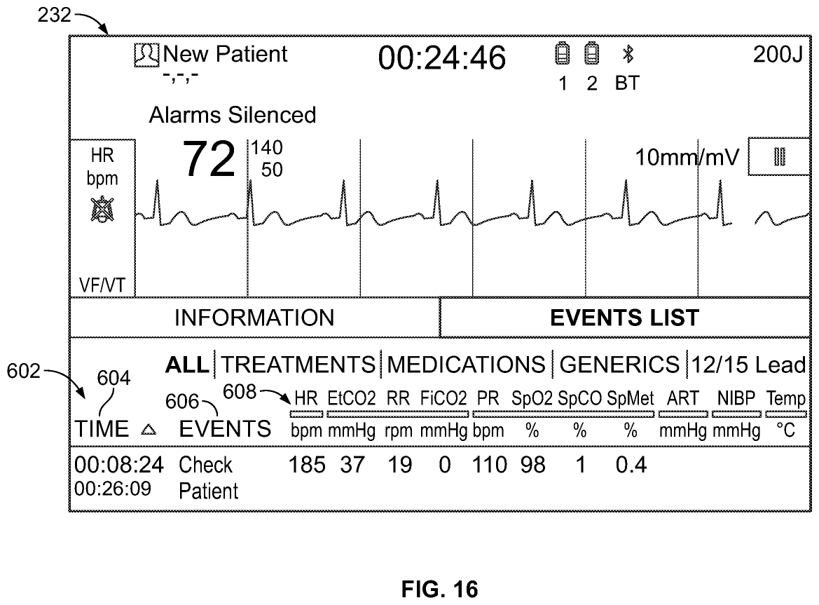

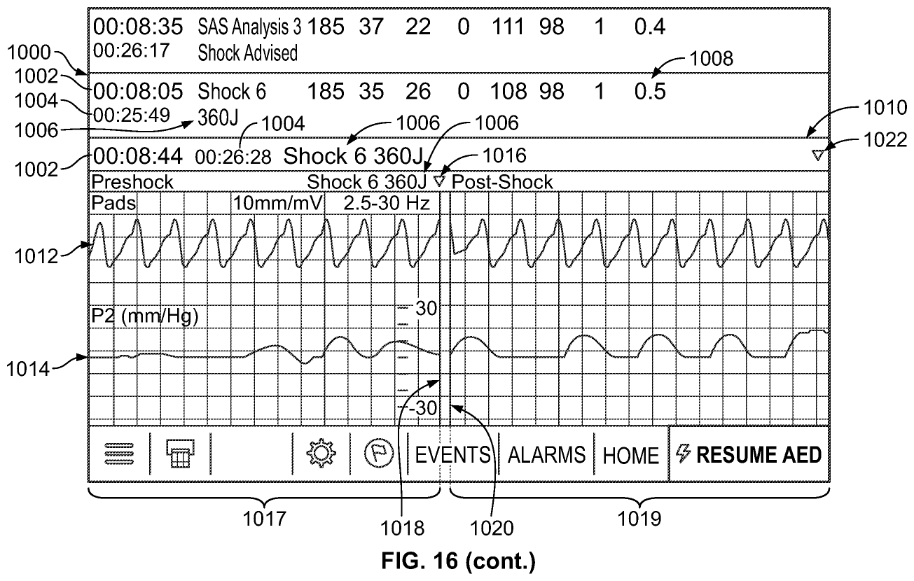

In addition to capturing the physiologic parameters of the patient when the event has occurred, the processor 302 of the defibrillator 104 obtains real-time data of one or more physiologic parameters (ECG, oxygen, blood pressure, etc.) before the event (e.g., within a time window of a particular period of time before the event such as 3-5 seconds), obtains real-time data of the one or more physiologic parameters after the event (e.g., within a time window of a particular period of time after the event such as 8 seconds), renders respective waveforms of the one or more physiologic parameters, and attaches or associates the respective waveforms to the event record. To view waveforms associated with an event, the healthcare professional can press anywhere in the row for that event. In an example, up to three waveforms can be displayed for each event depending on the type of event, as well as the configuration of the sensors and the defibrillator 104 at the time of the event. An example of a waveform associated with an event is described below with respect to FIG. 16.

Further, the events list view pane 600 includes an event list filter menu bar 610 having multiple tabs that facilitate filtering the list of events shown in the events list 602. For example, the event list filter menu bar 610 includes an All events tab 612, a Treatments tab 614, a Medications tab 616, a Generic events tab 618, and a 12/15 Lead tab 620. FIG. 6 illustrates the events list 602 when the All events tab 612 is selected. Selecting one of the tab filters the list of events such that the events list 602 displays only the events that pertain to the type of event of the respective tab (e.g., medications events, treatments events, generic events, 12/15 Lead ECG capturing events).

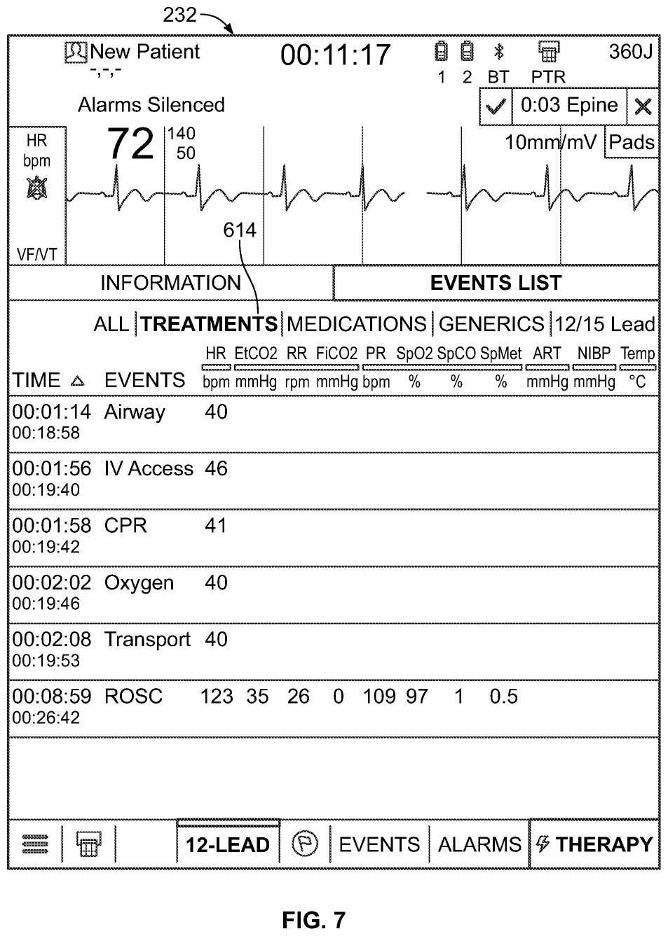

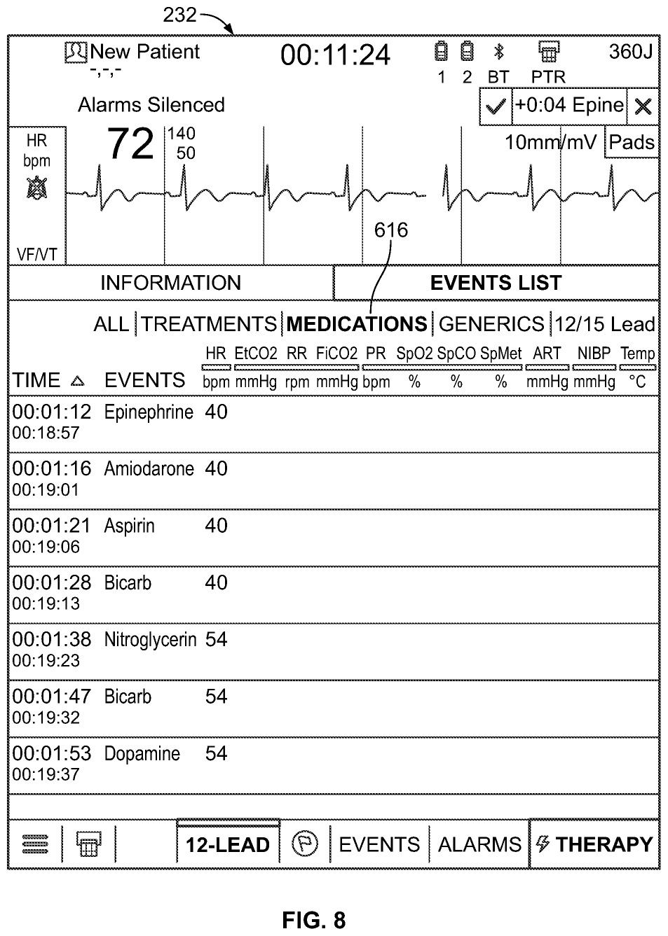

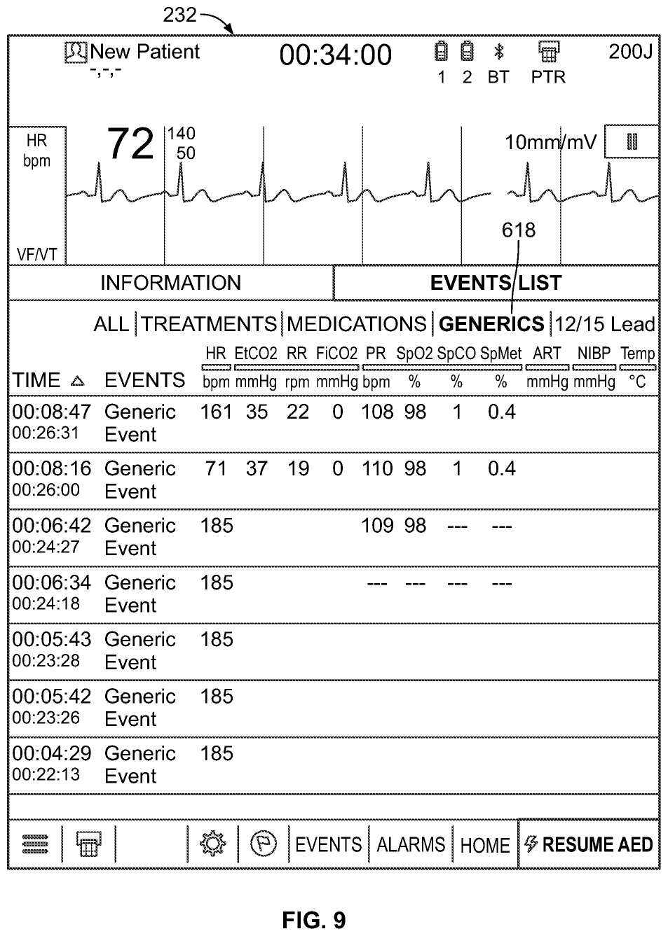

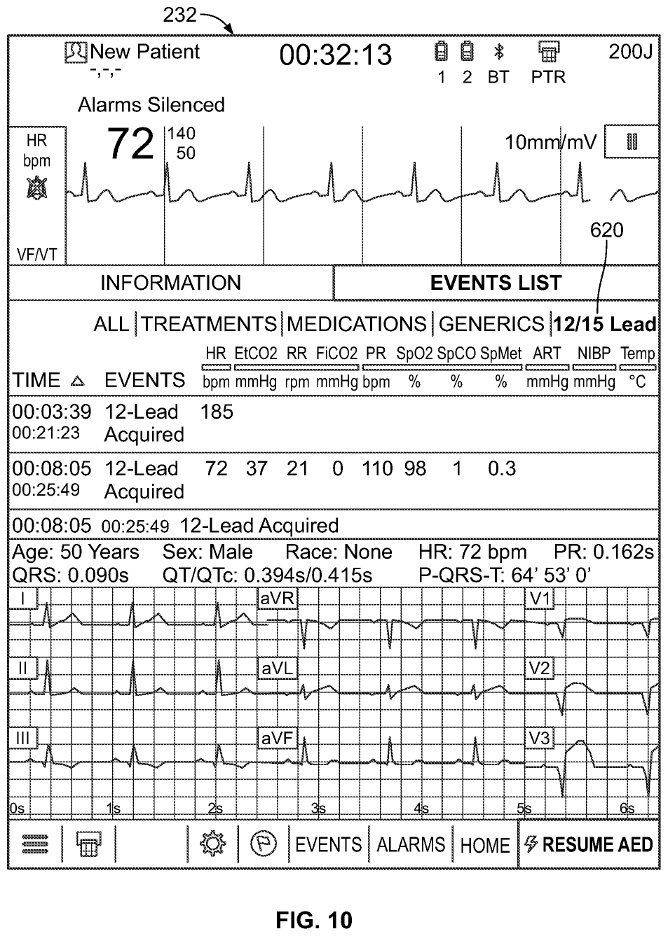

FIG. 7 illustrates the events list 602 when the Treatments tab 614 is selected, FIG. 8 illustrates the events list 602 when the Medications tab 616 is selected, FIG. 9 illustrates the events list 602 when the Generic events tab 618 is selected, and FIG. 10 illustrates the events list 602 when the 12/15 Lead tab 620 is selected, in accordance with an example implementation.

Events in the events list 602 can either be automatically detected or manually entered. For example, the processor 302 can detect some events automatically based on physiologic monitoring data captured when the events occur. An example event that the processor 302 can detect automatically is a shock event where the processor 302 causes the defibrillator 104 to automatically apply a shock to the patient upon detecting physiologic conditions, such as shockable heart rhythms, and making a decision based on an analysis of the patient's heart data to shock the patient's heart at a particular time. The processor 302 then automatically logs the shock event in the events list 602

Another example automatically-detected event is when the processor 302 detects that a physiologic parameter decreased below a threshold value (e.g., HR decreased below 50 beats per minute) or increase beyond a threshold value (e.g., FiCO2 increased above 8). As another example, the processor 302 can automatically capture an initial rhythm of the heart (e.g., initial ECG) at the beginning of a patient episode and automatically logs the Initial Rhythm event record 603 (see FIG. 6) in the event list 602.

Another example automatically-detected event is when the processor 302 determines that it is advised to shock the patient at a particular time and issues an alarm and/or logs a “Shock Advised” event in the events list 602. As another automatically-detected example, if a healthcare professional commands the defibrillator 104 to capture a 12 Lead ECG (e.g., by pressing the 12-Lead button 412 shown in FIG. 4), the processor 302 automatically logs the 12 Lead ECG event and associated data in the events list 602. As another example, the processor 302 can automatically detect a “pacing” event.

Additionally or alternatively, events can be added to the events list 602 manually. For example, to add a Generic event, the healthcare professional can press the Generic Event button 414. An example generic event is when the healthcare professional wants to capture heart rhythm and physiologic parameters of the patient at a particular point in time during the course of treating the patient in an on-going episode. Generic events might not include any text, but they can be annotated later if desired.

Another way to add events is through pressing the Events button 416. When the Events button 416 is pressed, an events menu appears that lists different types of events that can be added to the events list 602. The different types of events include for example, treatments and medications administered to the patient.

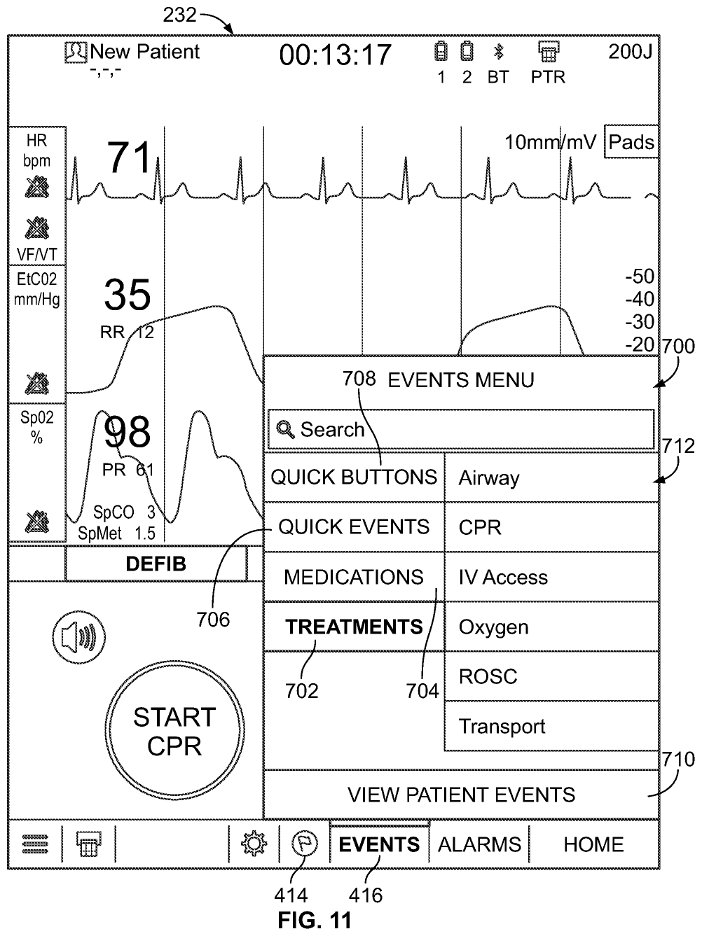

FIG. 11 illustrates an events menu 700 that appears when the Events button 416 is selected, in accordance with an example implementation. As shown, the events menu 700 includes four menu options: Treatments option 702, Medications option 704, Quick Events option 706, and Quick Buttons option 708. The events menu 700 also includes a View Patient Events option 710 that, when pressed, reverts the GUI 232 back to the view showing the events list 602.

FIG. 11 illustrates the events menu 700 when the Treatments option 702 is selected. As shown, to the right of the Treatments option 702 appears a Treatments menu 712 that has a scrollable list of treatments that the healthcare professional can chose from. The list of treatments can be customizable by an organization (e.g., the Hospital) that owns the defibrillator 104. An example list of treatments include Airway treatment, CPR treatment, Intravenous (IV) Access treatment (e.g., to administer fluids and medications), Oxygen treatment, ROSC treatment, and Transport events. The healthcare professional can select any of the listed treatments, and responsively the processor 302 adds an event for the particular treatment selected to the events list 602 and generates a corresponding event record with various captured physiologic parameters and waveforms.

When the Medications option 704 is selected a Medications menu appears to the right of the Medications option 704 appears a Medications menu that has a scrollable list of medications that the healthcare professional can chose from when a particular medication in the list is administered to the patient. The list of medications can be customizable by an organization (e.g., the Hospital) that owns the defibrillator 104. An example list of medications include Adenosine, Amiodarone, Aspirin, Atropine, Bicarb, Dopamine, Epinephrine, Glucose, Heparin, Lidocaine, Morphine, Naloxone, Nitroglycerin, Thrombolytic, and Vasopressin. The healthcare professional can select any of the listed medications, and responsively the processor 302 adds an event for the particular treatment selected to the events list 602 and generates a corresponding event record.

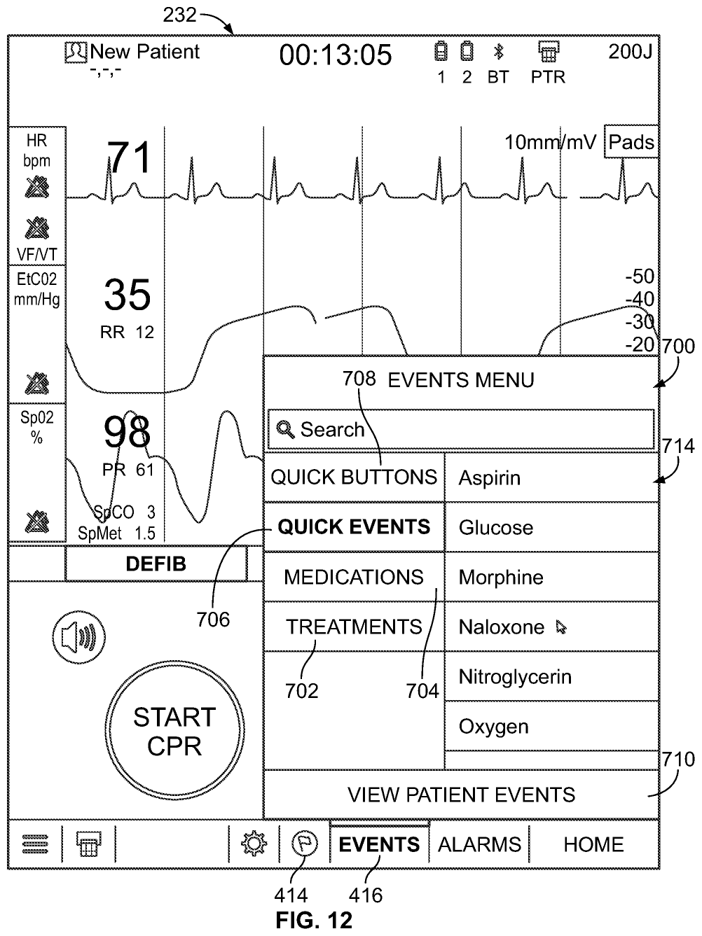

FIG. 12 illustrates the events menu 700 when the Quick Events option 706 is selected, in accordance with an example implementation. As shown, to the right of the Quick Events option 706 appears a Quick Events menu 714 that has a customizable list of the most frequently used Treatments and Medications. The list of quick events can be customizable by an organization (e.g., the Hospital) that owns the defibrillator 104. The healthcare professional can select any of the listed treatments, and responsively the processor 302 adds an event for the particular event selected to the events list 602 and generates a corresponding event record.

The Quick Events menu 714 includes events from the lists that are defined in the Medications menu and the Treatment menu 712. For example, if the Medications menu has a list of thirteen medications and the Treatments menu 712 has a list of six treatments, the Quick Events menu 714 may include a scrollable list of seven of the most commonly selected events form both the Medications menu and the Treatment menu 712.

Notably, if a healthcare professional edits or deletes a medication or treatment event that is included in the respective menu, the same change applies to the Quick Event menu 714.

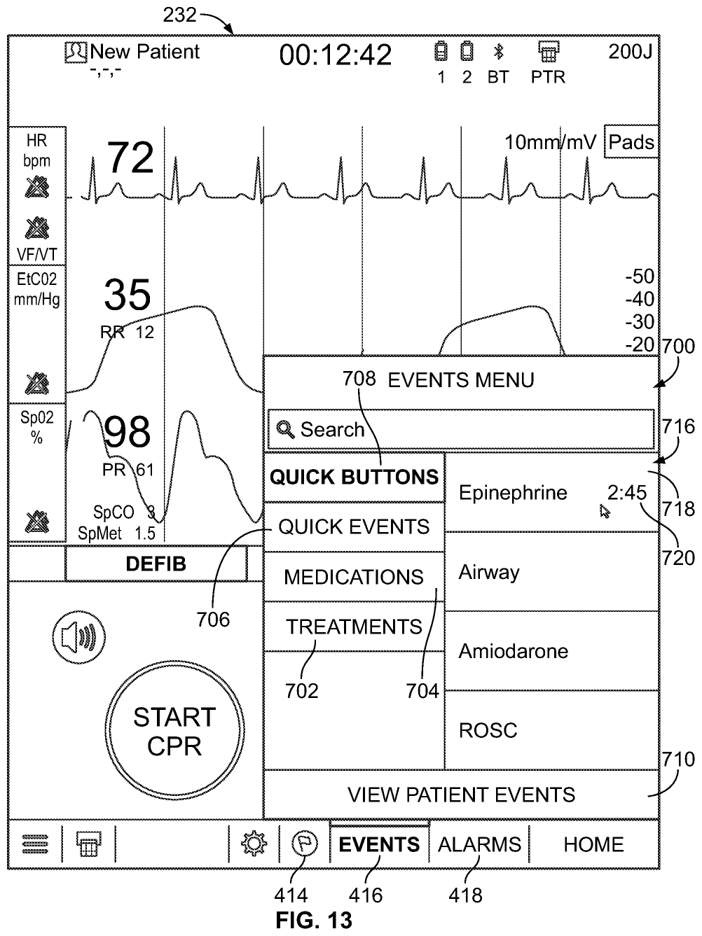

FIG. 13 illustrates the events menu 700 when the Quick Buttons option 708 is selected. As shown, to the right of the Quick Buttons option 708 appears a Quick Buttons menu 716 that has a customizable list of a particular number (e.g., four) of the most frequently used events. The list of events in the Quick Buttons menu 716 can be customizable by an organization (e.g., the Hospital) that owns the defibrillator 104. The healthcare professional can select any of the listed treatments, and responsively the processor 302 adds an event for the particular treatment or medication selected from the Quick Buttons menu 716 to the events list 602 and generates a corresponding event record.

The Quick Buttons menu 716 includes events from the lists that are defined in the Medications menu and the Treatment menu 712. For example, if the Medications menu has a list of thirteen medications and the Treatments menu 712 has a list of six treatments, the Quick Buttons menu 716 includes four the most commonly selected events form both the Medications menu and the Treatment menu 712 (e.g., Epinephrine medication event, Airway treatment event, Amiodarone medication event, and ROSC event).

The Quick Buttons menu 716 differs from the Quick Events menu 714 in that a timer function can be associated with the events that are selected from the Quick Buttons menu 716. Particularly, in addition to the button title of each of the events in the Quick Button menu 716, a timer function can be added if desired. For instance, as shown in FIG. 13, a Quick Event button 718 titled “Epinephrine” has a timer 720 that provides a reminder to repeat the therapy (i.e., repeat administering Epinephrine) after a specified period of time has passed. The period of time is customizable or configurable by the user based on the type of medication or event and the frequency with which it is to be repeated.

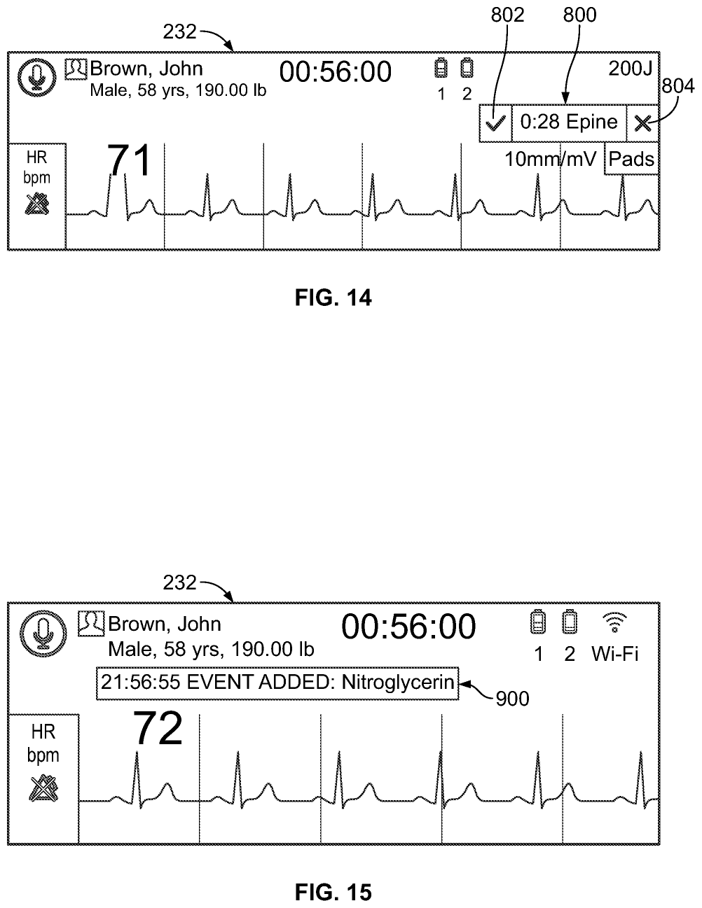

FIG. 14 illustrates a partial view of the GUI 232 showing a reminder display 800, in accordance with an example implementation. Quick Button events can be set up to have a single reminder after a certain time interval, or to have recurring reminders. The reminder display 800 can appear at the top of the GUI 232 at a predefined point in time (e.g., 30 seconds) before the timer expires and therapy is due to be repeated, for example. The reminder display 800 depicts a timer that counts down a particular period of time (e.g., the last 30 seconds) before a therapy (e.g., medication or treatment) is due.

To indicate that the therapy has been delivered, the healthcare professional can press a check mark button 802 in the reminder display 800. If the timer is set to be recurring, the reminder is repeated until the user dismisses it. To dismiss the reminder and stop recurring reminders, the user can press the “X” button 804 in the reminder display 800.

In an example, each time an event is added to the events list 602, a confirmation message appears on the GUI 232. FIG. 15 illustrates a partial view of the GUI 232 showing a notification 900 of an added event, in accordance with an example implementation. As depicted in FIG. 15, the notification 900 comprises a message showing a time stamp (i.e., chronological time) of when the event has been added as well as the name of the event: “Nitroglycerin,” for example. The notification 900 may remain on the GUI 232 for a particular period of time (e.g., for several seconds) and is then removed.

As mentioned above with respect to FIG. 6, for reach event added to the events list 602, the processor 302 obtains real-time data of one or more physiologic parameter (ECG, oxygen, blood pressure, etc.) within a particular period of time before the event (3 seconds) and obtains real-time data of the one or more physiologic parameter for a particular period of time (e.g., 8 seconds) after the event. The processor 302 then renders respective waveforms of the one or more physiologic parameter, and attaches the respective waveforms to the event record. To view waveforms associated with an event, the healthcare professional can press anywhere in the row for that event.

FIG. 16 illustrates the GUI 232 with waveforms associated with a shock event being displayed, in accordance with an example implementation. FIG. 16 is depicted on two drawing sheets to clearly depict elements of the Figure and reduce visual clutter.

An event record row 1000 of the shock event shows chronological time 1002 of when the shock has occurred and elapsed time 1004 since the shock has occurred. The event record row 1000 also shows an event name 1006 “Shock 6, 360J” of the event indicating the type of the event and the energy used in the shock in Joules. The event record row 1000 further shows physiologic parameter values 1008 corresponding to the physiologic parameter headings of the physiologic parameter columns 608.

A healthcare professional can press anywhere in the event record row 1000 to select that particular event record, and responsively the processor 302 generates a display of a waveform viewer 1010. The waveform viewer 1010 displays the chronological time 1002, the elapsed time 1004, and the event name 1006 again to facilitate identification of the event to which the waveforms pertain.

The waveform viewer 1010 shows a first waveform 1012 that traces HR or ECG data over time. The waveform viewer 1010 also shows and a second waveform 1014 that traces invasive blood pressure measurement over time. The number and types of waveforms displayed are based on the type of event, for example. As examples, for an Initial Rhythm event, one waveform of ECG data may be sufficient; for a 12 Lead event, three waveforms corresponding to the V1, V2, and V3 leads may be shown; for an ROSC event, waveforms corresponding to ECG data, blood pressure, and EtCO2 may be shown, and so forth. As such, in examples, up to three waveforms can be displayed depending on the type of event and the configuration of the physiologic monitoring sensors 320 and the defibrillator 104.

The waveform viewer 1010 further shows a Moment of Event icon 1016 depicted as a triangle or arrow head pointing downward to indicate a point in time where the shock is applied to the patient. As such, the Moment of Event icon 1016 separates a first portion 1017 of the waveforms 1012, 1014 captured before the event occurred (before the shock is applied) and a second portion 1019 of the waveform 1012 captured after the event occurred (after the shock is applied). This way, the healthcare professional can see the effect of the event on the state of the patient as indicated by the physiologic parameter represented by the waveform.

As such, the processor 302 is configured to store data associated with a physiologic parameter of a waveform in a data buffer. The data buffer can be in the memory 304 used to temporarily store data for a particular period of time (e.g., 3 seconds). This way, when an event occurs, the processor 302 adds the data captured after the event to the data in the data buffer so generate or render the waveforms 1012, 1014 and associate them with the event record of the event.

In some examples, as shown in FIG. 16, the waveform viewer 1010 can include a first window 1018 (e.g., a rectangle) that encompasses the first portion 1017 of the waveforms 1012, 1014 that is captured before the event. In those examples, the waveform viewer 1010 can also include a second window 1020 that encompasses the second portion 1019 of the waveforms 1012, 1014 that is captured after the event occurred.

In an example, the period of time of the first portion 1017 of the waveforms 1012, 1014 is the same as the respective period of time of the second portion 1019 of the waveforms 1012, 1014. In another example, the period of time of the first portion 1017 of the waveforms 1012, 1014 is different from the respective period of time of the second portion 1019 of the waveforms 1012, 1014. For instance, the first period of time can be 3 seconds while the second period of time is 8 seconds.

In an example, the waveforms 1012, 1014 are scrollable. Particularly, the waveforms 1012, 1014 can be horizontally-scrollable.

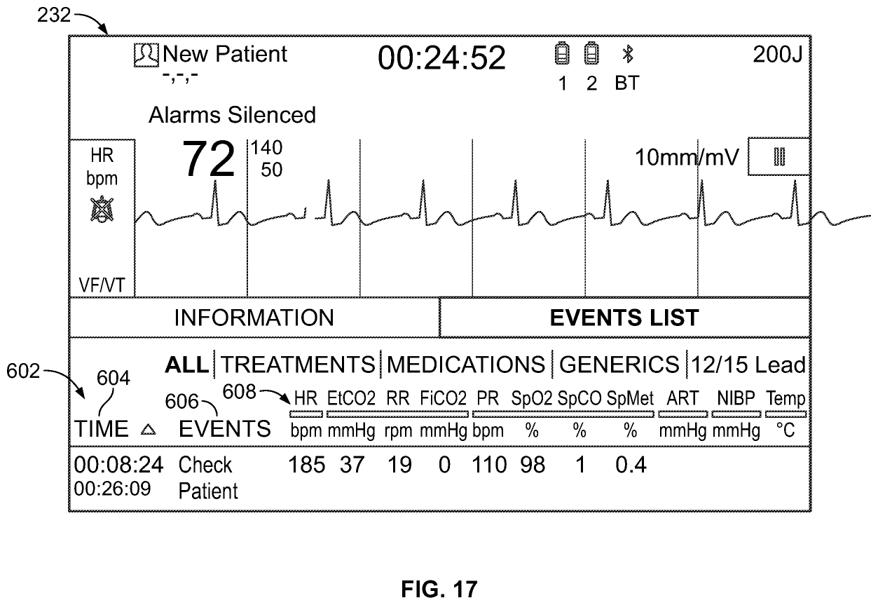

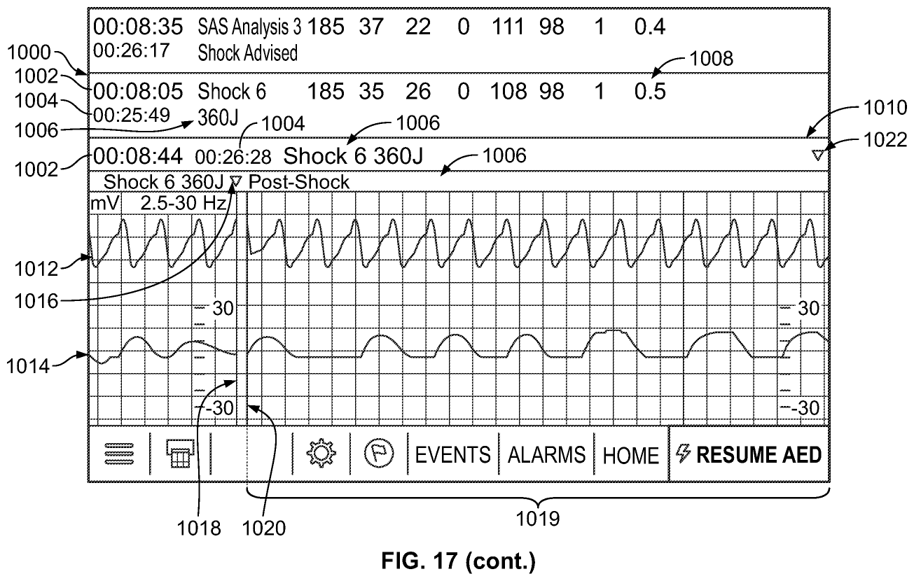

FIG. 17 illustrates horizontal scrolling of the waveforms 1012, 1014, in accordance with an example implementation. Similar to FIG. 16, FIG. 17 is also depicted on two drawing sheets to clearly depict elements of the Figure and reduce visual clutter.

The healthcare professional can scroll the waveforms 1012, 1014 horizontally to see more or less of first portion 1017 and the second portion 1019 of the waveforms 1012, 1014 as desired. For example, as shown in FIG. 17, the healthcare professional can scroll to the right to shown more of the second portion 1019 and less of the first portion 1017 to have an extended view of the waveforms 1012, 1014 after the event occurs.

The waveform viewer 1010 further includes a collapse button 1022 depicted as a triangle or arrow head pointing downward. When the collapse button 1022 is pressed, the waveform viewer 1010 is collapsed and the healthcare professional can then press on or select a different event record to display the waveforms associated with such different event record.

Thus, during and throughout an on-going patient episode, the defibrillator 104 provides an on-device real-time events review tools with physiologic parameters (e.g., vital signs) and waveform review capabilities, thus providing an on-device presentation of collected data and making the data available immediately during the episode. This way, a treating healthcare professional has continual access to history, medications doses, or any other events that has occurred with time stamps of each event in addition to various physiologic parameters and waveforms that have been captured during the event. Thus, a healthcare professional need not remember all the events or document the events while caring for the patient. Further, such methods and systems may help ease cognitive off-load of a paramedic or EMT through the handing-off or transition to a hospital or other treating facility.

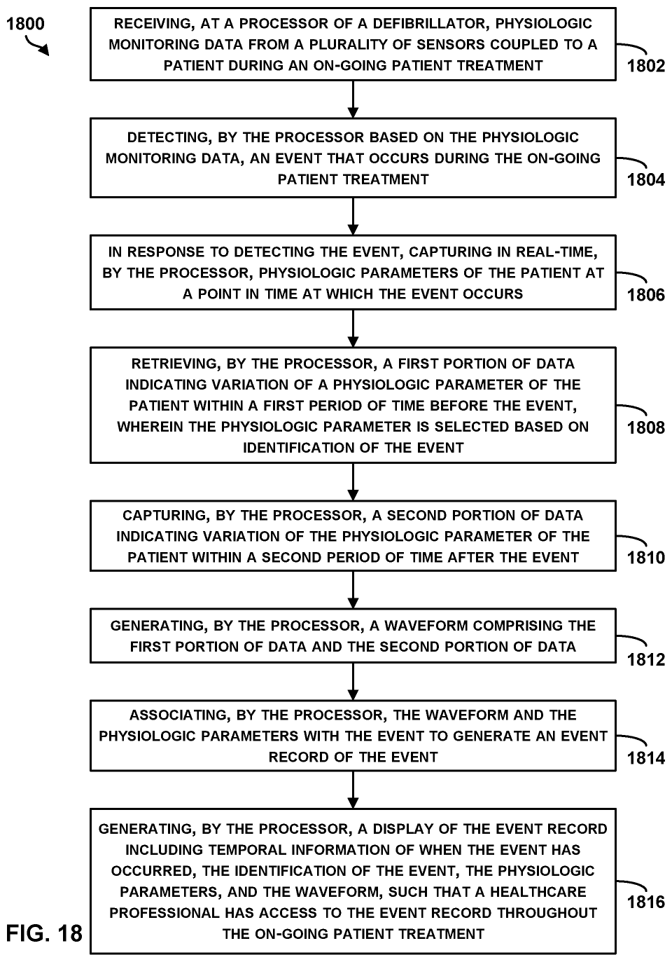

FIG. 18 is a flowchart of a method 1800 for operating the defibrillator 104, in accordance with an example implementation. Method 1800 shown in FIG. 18 presents an example of a method that could be used or implemented by the processor 302 of the defibrillator 104, for example. Further, devices or systems may be used or configured to perform logical functions presented in FIG. 18. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner. Method 1800 may include one or more operations, functions, or actions as illustrated by one or more of blocks 1802-1816. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

It should be understood that for this and other processes and methods disclosed herein, flowcharts show functionality and operation of one possible implementation of present examples. In this regard, each block or portions of each block may represent a module, a segment, or a portion of program code, which includes one or more instructions executable by a processor for implementing specific logical functions or steps in the process. The program code may be stored on any type of computer readable medium or data storage, for example, such as a storage device including a disk or hard drive. Further, the program code can be encoded on a computer-readable storage media in a machine-readable format, or on other non-transitory media or articles of manufacture. The computer readable medium may include non-transitory computer readable medium or memory, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a tangible computer readable storage medium, for example.

In addition, each block or portions of each block in FIG. 18, and within other processes and methods disclosed herein, may represent circuitry that is wired to perform the specific logical functions in the process. Alternative implementations are included within the scope of the examples of the present disclosure in which functions may be executed out of order from that shown or discussed, including substantially concurrent or in reverse order, depending on the functionality involved, as would be understood by those reasonably skilled in the art.

At block 1802, the method 1800 includes receiving, at the processor 302 of the defibrillator 104, physiologic monitoring data from a plurality of sensors (e.g., the physiologic monitoring sensors 320) coupled to the patient 102 during an on-going patient treatment.

At block 1804, the method 1800 includes detecting, by the processor 302 based on the physiologic monitoring data, an event that occurs during the on-going patient treatment. As described above, an event can be a treatment evet, a medications event, a generic event, a 12/15 Lead ECG capture event, etc. The processor 302 automatically detects that the event has occurred based on the physiologic monitoring data, or receives a request by the healthcare professional to add the event to the events list 602.

At block 1806, the method 1800 includes, in response to detecting the event, capturing in real-time, by the processor 302, physiologic parameters (e.g., HR, EtCO2, RR, FiCO2, PR, SpO2, SpCO, SpMet, NIBP, Temperature, etc.) of the patient at a point in time at which the event occurs.

At block 1808, the method 1800 includes retrieving, by the processor 302, a first portion of data (e.g., the first portion 1017) indicating variation of a physiologic parameter of the patient 102 within a first period of time (e.g., 3 seconds) before the event, wherein the physiologic parameter is selected based on identification of the event. As mentioned above, the processor 302 can determine up to three physiologic parameters associated with the event and can display up to three signals or waveforms depicting variation of the three physiologic parameters.

At block 1810, the method 1800 includes capturing, by the processor 302, a second portion of data (the second portion 1019) indicating variation of the physiologic parameter of the patient 102 within a second period of time (e.g., 8 seconds) after the event. In an example, the second period of time is greater than the first period of time.

At block 1812, the method 1800 includes generating, by the processor 302, a waveform (e.g., the waveform 1012, 1014) comprising the first portion of data and the second portion of data.

At block 1814, the method 1800 includes associating, by the processor 302, the waveform and the physiologic parameters with the event to generate an event record (e.g., the event record of the event record row 1000 from FIG. 16) of the event.

At block 1816, the method 1800 includes generating, by the processor 302, a display of the event record including temporal information of when the event has occurred, the identification of the event (e.g., the name of the event), the physiologic parameters, and the waveform, such that a healthcare professional has access to the event record throughout the on-going patient treatment.

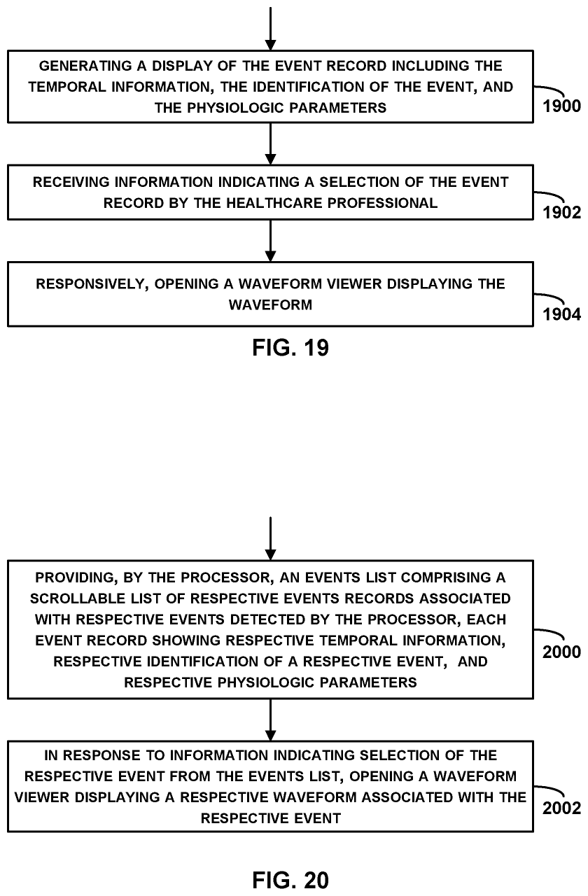

FIG. 19 is a flowchart of additional operations that are executable with the method 1800, in accordance with an example implementation. Generating a display of the event record can comprise several operations. At block 1900, the operations include generating a display of the event record including the temporal information, the identification of the event, and the physiologic parameters (without the waveform). At block 1902, the operations include receiving information indicating a selection of the event record by the healthcare professional. At block 1904, the operations include, responsively, opening the waveform viewer 1010 displaying the waveform.

FIG. 20 is a flowchart of additional operations that are executable with the method 1800, in accordance with an example implementation. At block 2000, the operations include providing, by the processor 302, an events list (e.g., the events list 602) comprising a scrollable list of respective events records associated with respective events detected by the processor 302, each event record showing respective temporal information, respective identification of a respective event, and respective physiologic parameters. At block 2002, the operations include, in response to information indicating selection of the respective event from the events list (e.g., a selection by the healthcare professional via the touchscreen 234 displaying the GUI 232, opening the waveform viewer 1010 displaying a respective waveform (e.g., the waveform 1012, 1014) associated with the respective event.

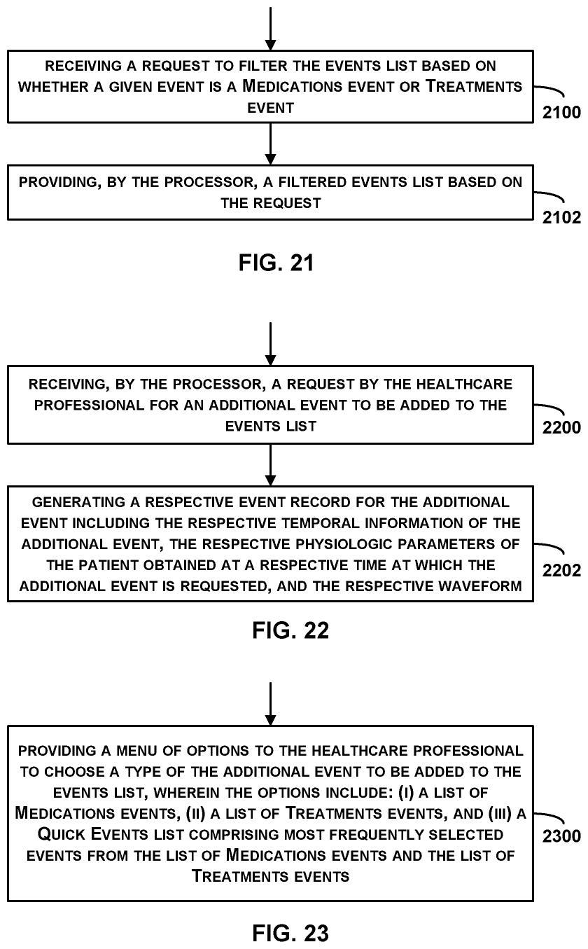

FIG. 21 is a flowchart of additional operations that are executable with the method 1800, in accordance with an example implementation. As described above, the respective events include Medications events associated with administering a medication to the patient 102 and Treatments events associated with applying a treatment to the patient 102. The events can also include Generic events and 12/15 Lead ECG events as described above. At block 2100, the operations include receiving a request to filter the events list 602 based on whether a given event is a Medications event or Treatments event. For example, the healthcare professional can select a tab from the event list filter menu bar 610 to filter the list of events. At block 2102, the operations include providing, by the processor 302, a filtered events list based on the request.

FIG. 22 is a flowchart of additional operations that are executable with the method 1800, in accordance with an example implementation. At block 2200, the operations include receiving, by the processor 302, a request by the healthcare professional for an additional event to be added to the events list 602. For example, the healthcare professional can select the Events button 416 to show the Event menu 700 and select the type of event that healthcare professional wants to add, then select the event from the menu (e.g., from the Treatments menu 712, the Medications menu, the Quick Events menu 714, or the Quick Buttons menu 716). At block 2202, the operations include generating a respective event record for the additional event including the respective temporal information of the additional event, the respective physiologic parameters of the patient obtained at a respective time at which the additional event is requested, and the respective waveform.

FIG. 23 is a flowchart of additional operations that are executable with the method 1800, in accordance with an example implementation. At block 2300, the operations include providing a menu of options to the healthcare professional to choose a type of the additional event to be added to the events list, wherein the options include: (i) a list of Medications events, (ii) a list of Treatments events (the Treatments menu 712), and (iii) a Quick Events list (the Quick Events menu 714) comprising most frequently selected events from the list of Medications events and the list of Treatments events.

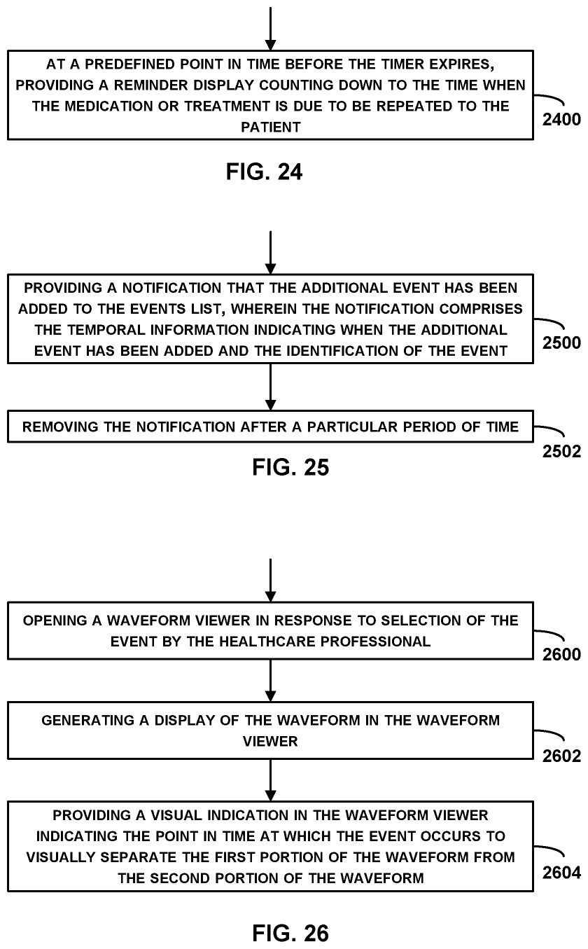

FIG. 24 is a flowchart of additional operations that are executable with the method 1800, in accordance with an example implementation. As mentioned above, the options can further include: a Quick Buttons list (e.g., the Quick Buttons menu 716) comprising most frequently selected events from the list of Medications events and the list of Treatments events, wherein each Medication event or Treatment event in the Quick Buttons list is associated with a timer (e.g., the timer 720) indicating a count-down to a time when a medication or treatment is due to be repeated to the patient 102. At block 2400, the operations include at a predefined point in time before the timer expires (e.g., 30 seconds before the timer expires), providing a reminder display (e.g., the reminder display 800) counting down to the time when the medication or treatment is due to be repeated to the patient 102.

FIG. 25 is a flowchart of additional operations that are executable with the method 1800, in accordance with an example implementation. At block 2500, the operations include providing a notification (e.g., the notification 900) that the additional event has been added to the events list 602, wherein the notification comprises the temporal information indicating when the additional event has been added and the identification of the event. At block 2502, the operations include removing the notification after a particular period of time (e.g., 2-5 seconds).

FIG. 26 is a flowchart of additional operations that are executable with the method 1800, in accordance with an example implementation. The operation of generating a display of the waveform can comprises several operations. At block 2600, the operations include opening the waveform viewer 1010 in response to selection of the event by the healthcare professional. At block 2602, the operations include generating a display of the waveform (e.g., the waveform 1012, 1014) in the waveform viewer 1010. At block 2604, the operations further include providing a visual indication (e.g., the Moment of Event icon 1016) in the waveform viewer 1010 indicating the point in time at which the event occurs to visually separate the first portion 1017 of the waveform from the second portion 1019 of the waveform.

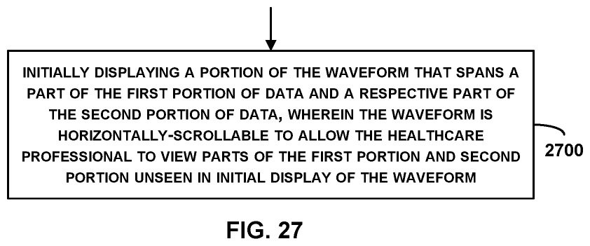

FIG. 27 is a flowchart of additional operations that are executable with the method 1800, in accordance with an example implementation. At block 2700, generating a display of the waveform in the waveform viewer comprises initially displaying a portion of the waveform that spans a part of the first portion 1017 of data and a respective part of the second portion 1019 of data, wherein the waveform is horizontally-scrollable to allow the healthcare professional to view parts of the first portion 1017 and second portion 1019 unseen in initial display of the waveform.

Implementations of this disclosure provide technological improvements that are particular to defibrillators, for example, those concerning detecting events that occur during an on-going patient treatment episode, capturing physiologic parameter information as the event occurs, and generating waveforms shown variation of one or more physiologic parameters before and after the event. Thus, defibrillator-specific technological problems, such as detecting events, capturing associated information, and having access to all such events and information captured by the defibrillator throughout patient treatment can be wholly or partially solved by implementations of this disclosure. Implementations of this disclosure can thus introduce new and efficient improvements in the ways in which events are processed by, and made available via, defibrillators.

Further, the disclosure provides a graphical user interface that enables on-device real-time patient events review tools with physiological parameters (e.g., vital signs) and waveform review capabilities, thus providing an on-device presentation of collected data and making the data available immediately during an emergency episode. This way, a treating healthcare professional has continual access to history, medications doses, or any other events that has occurred with time stamps of each event in addition to various physiologic parameters and waveforms that have been captured during the event. Thus, a healthcare professional need not remember all the events or document the events while caring for the patient. Further, such methods and systems may help ease cognitive off-load of a paramedic or EMT through the handing-off or transition to a hospital or other treating facility.

The detailed description above describes various features and operations of the disclosed systems with reference to the accompanying figures. The illustrative implementations described herein are not meant to be limiting. Certain aspects of the disclosed systems can be arranged and combined in a wide variety of different configurations, all of which are contemplated herein.

Further, unless context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

Further, devices or systems may be used or configured to perform functions presented in the figures. In some instances, components of the devices and/or systems may be configured to perform the functions such that the components are actually configured and structured (with hardware and/or software) to enable such performance. In other examples, components of the devices and/or systems may be arranged to be adapted to, capable of, or suited for performing the functions, such as when operated in a specific manner.

By the term “substantially” or “about” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

The arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. Also, the terminology used herein is for the purpose of describing particular implementations only, and is not intended to be limiting.

Embodiments of the present disclosure can thus relate to one of the enumerated example embodiment (EEEs) listed below.

EEE 1 is a method comprising: receiving, at a processor of a defibrillator, physiologic monitoring data from a plurality of sensors coupled to a patient during an on-going patient treatment; detecting, by the processor based on the physiologic monitoring data, an event that occurs during the on-going patient treatment; in response to detecting the event, capturing in real-time, by the processor, physiologic parameters of the patient at a point in time at which the event occurs; retrieving, by the processor, a first portion of data indicating variation of a physiologic parameter of the patient within a first period of time before the event, wherein the physiologic parameter is selected based on an identification of the event; capturing, by the processor, a second portion of data indicating variation of the physiologic parameter of the patient within a second period of time after the event; generating, by the processor, a waveform comprising the first portion of data and the second portion of data; associating, by the processor, the waveform and the physiologic parameters with the event to generate an event record of the event; and generating, by the processor, a display of the event record including temporal information of when the event has occurred, the identification of the event, the physiologic parameters, and the waveform, such that a healthcare professional has access to the event record throughout the on-going patient treatment.

EEE 2 is the method of EEE 1, wherein generating a display of the event record comprises: generating a display of the event record including the temporal information, the identification of the event, and the physiologic parameters; receiving information indicating a selection of the event record by the healthcare professional; and responsively, opening a waveform viewer displaying the waveform.

EEE 3 is the method of any of EEEs 1-2, further comprising: providing, by the processor, an events list comprising a scrollable list of respective events records associated with respective events detected by the processor, each event record showing respective temporal information, respective identification of a respective event, and respective physiologic parameters; and in response to information indicating selection of the respective event from the events list, opening a waveform viewer displaying a respective waveform associated with the respective event.

EEE 4 is the method of EEE 3, wherein the respective events include Medications events associated with administering a medication to the patient and Treatments events associated with applying a treatment to the patient, the method further comprising: receiving a request to filter the events list based on whether a given event is a Medications event or Treatments event; and providing, by the processor, a filtered events list based on the request.

EEE 5 is the method of any of EEEs 3-4, further comprising: receiving, by the processor, a request by the healthcare professional for an additional event to be added to the events list; and generating a respective event record for the additional event including the respective temporal information of the additional event, the respective physiologic parameters of the patient obtained at a respective time at which the additional event is requested, and the respective waveform.

EEE 6 is the method of EEE 5, further comprising: providing a menu of options to the healthcare professional to choose a type of the additional event to be added to the events list, wherein the options include: (i) a list of Medications events, (ii) a list of Treatments events, and (iii) a Quick Events list comprising most frequently selected events from the list of Medications events and the list of Treatments events.

EEE 7 is the method of EEE 6, wherein the options further include: a Quick Buttons list comprising most frequently selected events from the list of Medications events and the list of Treatments events, wherein each Medication event or Treatment event in the Quick Buttons list is associated with a timer indicating a count-down to a time when a medication or treatment is due to be repeated to the patient.

EEE 8 is the method of EEE 7, further comprising: at a predefined point in time before the timer expires, providing a reminder display counting down to the time when the medication or treatment is due to be repeated to the patient.

EEE 9 is the method of any of EEEs 5-8, further comprising: providing a notification that the additional event has been added to the events list, wherein the notification comprises the temporal information indicating when the additional event has been added and the identification of the event; and removing the notification after a particular period of time.

EEE 10 is the method of any of EEEs 1-9, wherein generating a display of the waveform comprises: opening a waveform viewer in response to selection of the event by the healthcare professional; and generating a display of the waveform in the waveform viewer, wherein the method further comprises: providing a visual indication in the waveform viewer indicating the point in time at which the event occurs to visually separate the first portion of the waveform from the second portion of the waveform.

EEE 11 is the method of EEE 10, wherein generating a display of the waveform in the waveform viewer comprises: initially displaying a portion of the waveform that spans a part of the first portion of data and a respective part of the second portion of data, wherein the waveform is horizontally-scrollable to allow the healthcare professional to view parts of the first portion and second portion unseen in initial display of the waveform.

EEE 12 is the method of any of EEEs 1-10, wherein the second period of time is greater than the first period of time.

EEE 13 is a non-transitory computer-readable medium having stored therein a plurality of executable instructions that, when executed by a processor of a defibrillator, causes the processor to perform operations comprising: detecting, based on physiologic monitoring data received from a plurality of sensors coupled to a patient during an on-going patient treatment, a plurality of events that occur during the on-going patient treatment; for each event detected: in response to detecting the event, capturing in real-time physiologic parameters of the patient at a point in time at which the event occurs, retrieving a first portion of data indicating variation of a physiologic parameter of the patient within a first period of time before the event, wherein the physiologic parameter is selected based on an identification of the event, capturing a second portion of data indicating variation of the physiologic parameter of the patient within a second period of time after the event, generating a waveform comprising the first portion of data and the second portion of data, associating the waveform and the physiologic parameters with the event to generate an event record of the event, and generating an event record including temporal information of when the event has occurred, the identification of the event, the physiologic parameters at the point in time at which the event occurs, and the waveform; providing an events list comprising a scrollable list of respective events records associated with respective events detected by the processor, each event record showing respective temporal information, respective identification of a respective event, and respective physiologic parameters such that a healthcare professional has access to the events records throughout the on-going patient treatment; and in response to information indicating selection of a particular event record from the events list, opening a waveform viewer displaying a respective waveform associated with the respective event.

EEE 14 is the non-transitory computer-readable medium of EEE 13, wherein the respective events include Medications events associated with administering a medication to the patient and Treatments events associated with applying a treatment to the patient, wherein the operations further comprise: receiving a request to filter the events list based on whether a given event is a Medications event or a Treatments event; and providing, by the processor, a filtered events list based on the request.

EEE 15 is the non-transitory computer-readable medium of any of EEEs 13-14, wherein detecting that an event has occurred comprises: automatically detecting that the event has occurred based on the physiologic monitoring data, or receiving a request by the healthcare professional to add the event to the events list.

EEE 16 is the non-transitory computer-readable medium of EEE 15, wherein the operations further comprise: providing a menu of options to the healthcare professional to choose a type of the event to be added to the events list, wherein the options include: (i) a list of Medications events, (ii) a list of Treatments events, (iii) a Quick Events list comprising most frequently selected events from the list of Medications events and the list of Treatments events, and (iv) a Quick Buttons list comprising most frequently selected events from the list of Medications events and the list of Treatments events, wherein each Medication event or Treatment event in the Quick Buttons list is associated with a timer indicating a count-down to a time when a medication or treatment is due to be repeated to the patient, and wherein receiving the request by the healthcare professional is based on a selection of the event from the list of Medications events, the list of Treatments events, the Quick Events list, or the Quick Buttons list.

EEE 17 is a defibrillator comprising: a non-transitory computer-readable medium having stored therein a plurality of executable instructions; and a processor adapted to execute the plurality of executable instructions to perform operations comprising: detecting, based on physiologic monitoring data received from a plurality of sensors coupled to a patient during an on-going patient treatment, a plurality of events that occur during the on-going patient treatment, for each event detected: in response to detecting the event, capturing in real-time physiologic parameters of the patient at a point in time at which the event occurs, retrieving a first portion of data indicating variation of a physiologic parameter of the patient within a first period of time before the event, wherein the physiologic parameter is selected based on an identification of the event. capturing a second portion of data indicating variation of the physiologic parameter of the patient within a second period of time after the event, generating a waveform comprising the first portion of data and the second portion of data, associating the waveform and the physiologic parameters with the event to generate an event record of the event, and generating an event record including temporal information of when the event has occurred, identification of the event, the physiologic parameters at a time when the event occurs, and the waveform, generating a display of an events list comprising a scrollable list of respective events records associated with respective events detected by the processor, each event record showing respective temporal information, respective identification of a respective event, and respective physiologic parameters such that a healthcare professional has access to the events records throughout the on-going patient treatment, and in response to information indicating selection of a particular event record from the events list, opening a waveform viewer displaying a respective waveform associated with the respective event.

EEE 18 is the defibrillator of EEE 17, wherein the respective events include Medications events associated with administering a medication to the patient and Treatments events associated with applying a treatment to the patient, wherein the operations further comprise: receiving a request to filter the events list based on whether a given event is a Medications event or a Treatments event; and providing, by the processor, a filtered events list based on the request.

EEE 19 is the defibrillator of any of EEEs 17-18, wherein detecting that an event has occurred comprises: automatically detecting that the event has occurred based on the physiologic monitoring data, or receiving a request by the healthcare professional to add the event to the events list.

EEE 20 is the defibrillator of EEE 19, wherein the operations further comprise: providing a menu of options to the healthcare professional to choose a type of the event to be added to the events list, wherein the options include: (i) a list of Medications events, (ii) a list of Treatments events, (iii) a Quick Events list comprising most frequently selected events from the list of Medications events and the list of Treatments events, and (iv) a Quick Buttons list comprising most frequently selected events from the list of Medications events and the list of Treatments events, wherein each Medication event or Treatment event in the Quick Buttons list is associated with a timer indicating a count-down to a time when a medication or treatment is due to be repeated to the patient, and wherein receiving the request by the healthcare professional is based on a selection of the event from the list of Medications events, the list of Treatments events, the Quick Events list, or the Quick Buttons list.

What is claimed is:

1. A method comprising:
receiving, at a processor of a defibrillator, physiologic monitoring data from a plurality of sensors coupled to a patient during an on-going patient treatment;
detecting, by the processor based on the physiologic monitoring data, an event that occurs during the on-going patient treatment;
in response to detecting the event, capturing in real-time, by the processor, physiologic parameters of the patient at a point in time at which the event occurs;
retrieving, by the processor, a first portion of data indicating variation of a physiologic parameter of the patient within a first period of time before the event, wherein the physiologic parameter is selected based on an identification of the event;
capturing, by the processor, a second portion of data indicating variation of the physiologic parameter of the patient within a second period of time after the event;
generating, by the processor, a waveform comprising the first portion of data and the second portion of data;
associating, by the processor, the waveform and the physiologic parameters with the event to generate an event record of the event; and
generating, by the processor, a display of the event record including temporal information of when the event has occurred, the identification of the event, the physiologic parameters, and the waveform, such that a healthcare professional has access to the event record throughout the on-going patient treatment.

2. The method of claim 1, wherein generating a display of the event record comprises:
generating a display of the event record including the temporal information, the identification of the event, and the physiologic parameters;
receiving information indicating a selection of the event record by the healthcare professional; and
responsively, opening a waveform viewer displaying the waveform.

3. The method of claim 1, further comprising:
providing, by the processor, an events list comprising a scrollable list of respective events records associated with respective events detected by the processor, each event record showing respective temporal information, respective identification of a respective event, and respective physiologic parameters; and
in response to information indicating selection of the respective event from the events list, opening a waveform viewer displaying a respective waveform associated with the respective event.

4. The method of claim 3, wherein the respective events include Medications events associated with administering a medication to the patient and Treatments events associated with applying a treatment to the patient, the method further comprising:
receiving a request to filter the events list based on whether a given event is a Medications event or Treatments event; and
providing, by the processor, a filtered events list based on the request.

5. The method of claim 3, further comprising:
receiving, by the processor, a request by the healthcare professional for an additional event to be added to the events list; and
generating a respective event record for the additional event including the respective temporal information of the additional event, the respective physiologic parameters of the patient obtained at a respective time at which the additional event is requested, and the respective waveform.

6. The method of claim 5, further comprising:
providing a menu of options to the healthcare professional to choose a type of the additional event to be added to the events list, wherein the options include: (i) a list of Medications events, (ii) a list of Treatments events, and (iii) a Quick Events list comprising most frequently selected events from the list of Medications events and the list of Treatments events.

7. The method of claim 6, wherein the options further include: a Quick Buttons list comprising most frequently selected events from the list of Medications events and the list of Treatments events, wherein each Medication event or Treatment event in the Quick Buttons list is associated with a timer indicating a count-down to a time when a medication or treatment is due to be repeated to the patient.

8. The method of claim 7, further comprising:
at a predefined point in time before the timer expires, providing a reminder display counting down to the time when the medication or treatment is due to be repeated to the patient.

9. The method of claim 5, further comprising:
providing a notification that the additional event has been added to the events list, wherein the notification comprises the temporal information indicating when the additional event has been added and the identification of the event; and
removing the notification after a particular period of time.

10. The method of claim 1, wherein generating a display of the waveform comprises:
opening a waveform viewer in response to selection of the event by the healthcare professional; and
generating a display of the waveform in the waveform viewer, wherein the method further comprises:
providing a visual indication in the waveform viewer indicating the point in time at which the event occurs to visually separate the first portion of the waveform from the second portion of the waveform.

11. The method of claim 10, wherein generating a display of the waveform in the waveform viewer comprises:
initially displaying a portion of the waveform that spans a part of the first portion of data and a respective part of the second portion of data, wherein the waveform is horizontally-scrollable to allow the healthcare professional to view parts of the first portion and second portion unseen in initial display of the waveform.

12. The method of claim 1, wherein the second period of time is greater than the first period of time.

13. A non-transitory computer-readable medium having stored therein a plurality of executable instructions that, when executed by a processor of a defibrillator, causes the processor to perform operations comprising:
detecting, based on physiologic monitoring data received from a plurality of sensors coupled to a patient during an on-going patient treatment, a plurality of events that occur during the on-going patient treatment;
for each event detected:
in response to detecting the event, capturing in real-time physiologic parameters of the patient at a point in time at which the event occurs,
retrieving a first portion of data indicating variation of a physiologic parameter of the patient within a first period of time before the event, wherein the physiologic parameter is selected based on an identification of the event,
capturing a second portion of data indicating variation of the physiologic parameter of the patient within a second period of time after the event,
generating a waveform comprising the first portion of data and the second portion of data,
associating the waveform and the physiologic parameters with the event to generate an event record of the event, and
generating an event record including temporal information of when the event has occurred, the identification of the event, the physiologic parameters at the point in time at which the event occurs, and the waveform;
providing an events list comprising a scrollable list of respective events records associated with respective events detected by the processor, each event record showing respective temporal information, respective identification of a respective event, and respective physiologic parameters such that a healthcare professional has access to the events records throughout the on-going patient treatment; and
in response to information indicating selection of a particular event record from the events list, opening a waveform viewer displaying a respective waveform associated with the respective event.

14. The non-transitory computer-readable medium of claim 13, wherein the respective events include Medications events associated with administering a medication to the patient and Treatments events associated with applying a treatment to the patient, wherein the operations further comprise:
receiving a request to filter the events list based on whether a given event is a Medications event or a Treatments event; and
providing, by the processor, a filtered events list based on the request.

15. The non-transitory computer-readable medium of claim 13, wherein detecting that an event has occurred comprises:
automatically detecting that the event has occurred based on the physiologic monitoring data, or receiving a request by the healthcare professional to add the event to the events list.

16. The non-transitory computer-readable medium of claim 15, wherein the operations further comprise:
providing a menu of options to the healthcare professional to choose a type of the event to be added to the events list, wherein the options include: (i) a list of Medications events, (ii) a list of Treatments events, (iii) a Quick Events list comprising most frequently selected events from the list of Medications events and the list of Treatments events, and (iv) a Quick Buttons list comprising most frequently selected events from the list of Medications events and the list of Treatments events, wherein each Medication event or Treatment event in the Quick Buttons list is associated with a timer indicating a count-down to a time when a medication or treatment is due to be repeated to the patient, and wherein receiving the request by the healthcare professional is based on a selection of the event from the list of Medications events, the list of Treatments events, the Quick Events list, or the Quick Buttons list.

17. A defibrillator comprising:
a non-transitory computer-readable medium having stored therein a plurality of executable instructions; and
a processor adapted to execute the plurality of executable instructions to perform operations comprising:
detecting, based on physiologic monitoring data received from a plurality of sensors coupled to a patient during an on-going patient treatment, a plurality of events that occur during the on-going patient treatment,
for each event detected:
in response to detecting the event, capturing in real-time physiologic parameters of the patient at a point in time at which the event occurs,
retrieving a first portion of data indicating variation of a physiologic parameter of the patient within a first period of time before the event, wherein the physiologic parameter is selected based on an identification of the event,
capturing a second portion of data indicating variation of the physiologic parameter of the patient within a second period of time after the event,
generating a waveform comprising the first portion of data and the second portion of data,
associating the waveform and the physiologic parameters with the event to generate an event record of the event, and
generating an event record including temporal information of when the event has occurred, identification of the event, the physiologic parameters at a time when the event occurs, and the waveform,
generating a display of an events list comprising a scrollable list of respective events records associated with respective events detected by the processor, each event record showing respective temporal information, respective identification of a respective event, and respective physiologic parameters such that a healthcare professional has access to the events records throughout the on-going patient treatment, and
in response to information indicating selection of a particular event record from the events list, opening a waveform viewer displaying a respective waveform associated with the respective event.

18. The defibrillator of claim 17, wherein the respective events include Medications events associated with administering a medication to the patient and Treatments events associated with applying a treatment to the patient, wherein the operations further comprise:
receiving a request to filter the events list based on whether a given event is a Medications event or a Treatments event; and
providing, by the processor, a filtered events list based on the request.

19. The defibrillator of claim 17, wherein detecting that an event has occurred comprises:
automatically detecting that the event has occurred based on the physiologic monitoring data, or receiving a request by the healthcare professional to add the event to the events list.

20. The defibrillator of claim 19, wherein the operations further comprise:
providing a menu of options to the healthcare professional to choose a type of the event to be added to the events list, wherein the options include: (i) a list of Medications events, (ii) a list of Treatments events, (iii) a Quick Events list comprising most frequently selected events from the list of Medications events and the list of Treatments events, and (iv) a Quick Buttons list comprising most frequently selected events from the list of Medications events and the list of Treatments events, wherein each Medication event or Treatment event in the Quick Buttons list is associated with a timer indicating a count-down to a time when a medication or treatment is due to be repeated to the patient, and wherein receiving the request by the healthcare professional is based on a selection of the event from the list of Medications events, the list of Treatments events, the Quick Events list, or the Quick Buttons list.
